(19) United States
(12) Patent Application Publication
(10) Pub. No.: US 2007/0203220 A1
(43) Pub. Date: Aug. 30, 2007

(54) Inhibitors Of PAI-1 For Treatment Of Muscular Conditions

(75) Inventors: David LeRoy Crandall, Doylestown, PA (US); George Phillip Vlasuk, Concord, MA (US)

Correspondence Address: WOODCOCK WASHBURN LLP/WYETH, CIRA CENTRE, 12TH FLOOR, 2929 ARCH STREET, PHILADELPHIA, PA, 19104-2891, US

(73) Assignee: Wyeth, Madison, NJ (US)

(21) Appl. No.: 11/679,031

(22) Filed: Feb. 26, 2007

Related U.S. Application Data
(60) Provisional application No. 60/777,521, filed on Feb. 27, 2006.

(51) Int. Cl. A61K 31/405

(52) U.S. Cl. 514/419

(57) ABSTRACT

This invention describes novel methods of treating muscle damage, muscle wasting, muscle degeneration, muscle atrophy or reduced rates of muscle repair associated with various conditions such as muscular dystrophy, through the use of small-molecule PAI-1 inhibitors.

### CROSS REFERENCE TO RELATED APPLICATION

This application claims the benefits of U.S. Provisional Application No. 60/777,521, filed Feb. 27, 2006, which is incorporated herein by reference in its entirety.

### FIELD

This invention describes novel methods of treating muscle damage, muscle wasting, muscle degeneration, muscle atrophy or reduced rates of muscle repair associated with various conditions such as muscular dystrophy, through the use of small-molecule PAI-1 inhibitors.

### BACKGROUND

There are currently few treatments for deleterious condition of the muscle, including, for example, muscle damage, muscle wasting, muscle degeneration, muscle atrophy or reduced rates of muscle repair. Such deleterious conditions of the muscle can result from normal conditions of use or trauma, or quite frequently, through chronic disease states. One such chronic disease state with very serious implications and of particular relevance to this invention is muscular dystrophy, a severe genetic disease associated with muscle wasting that has separate and diverse forms. Duchenne muscular dystrophy (DMD) results from mutations in the dystrophin protein, which ultimately leads to severe skeletal muscle wasting and death in early adulthood. Cardiomyopathy is also observed in DMD. The precise mechanisms for the progression of the disease are unknown, however, and treatment modalities have not been adequately developed. The outlook for any given muscular dystrophy sufferer is correlated with the degree of the severity of their disease. Some may live a normal lifespan and suffer from moderate symptoms while those afflicted with more severe forms of the disease can face a considerably bleaker outlook. Typical treatment modalities include rehabilitative exercises, physical therapy and the like. Corrective orthopedic surgery is employed in some instances and glucocorticoids may be used to prevent muscle wasting in DMD, but chronic administration of such agents are associated with a plethora of well-documented side effects. Clearly there is a current and urgent need for new treatments for this debilitating disease. This invention addresses this and other important ends.

### SUMMARY OF THE INVENTION

This invention provides, inter alia, methods of treating muscle damage, muscle wasting, muscle degeneration, muscle atrophy or reduced rate of muscle repair comprising administering an effective amount of a compound of a PAI-inhibitor, as provided herein, to a mammal in need thereof.

The invention also provides, inter alia, pharmaceutical compositions useful for treating muscle damage, muscle wasting, muscle degeneration, muscle atrophy or reduced rate of muscle repair comprising a PAI-inhibitor, as provided herein, and a pharmaceutically acceptable excipient.

The invention also provides, inter alia, uses of compounds and pharmaceutical compositions of the present invention in the manufacture of a medicament for treating muscle damage, muscle wasting, muscle degeneration, muscle atrophy or reduced rate of muscle repair.

### DESCRIPTION OF ILLUSTRATIVE EMBODIMENTS

The present invention provides, inter alia, methods of treating muscle damage, muscle wasting, muscle degeneration, muscle atrophy or reduced rate of muscle repair comprising administering a PAI-1 inhibitor to a mammal in need thereof.

In some embodiments, the PAI-1 inhibitors useful for the methods of this invention have a molecular weight of less than 1,000.

In some aspects, the PAI-1 inhibitors useful in the methods of this invention are described in US20060014725, US20050215626, US20050119327, US20050119326, US20050119296, US20050113439, US20050113438, US20050113438, US20050113436, US20050113428, US20050096377, US20050070592, US20050070587, US20050070585, US20050070584, US20040266733, US20040138283, US20040122070, US20040116504, US20040116488, US20030125371, US20030045560, US20030032626, US20030018067, and US20030013732, which are herein incorporated by reference in their entirety and for all purposes.

In some aspects, this invention describes a method of treating muscle damage, muscle wasting, muscle degeneration, muscle atrophy or reduced rate of muscle repair, wherein said method comprises the administration of an effective amount of a compound of formula (I), or a pharmaceutically acceptable salt, solvate or ester thereof, to a mammal in need thereof: 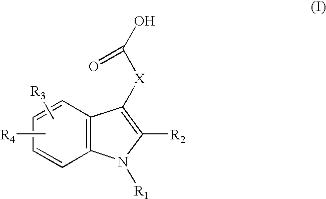
wherein:
X is a chemical bond, —CH₂— or —C(O)—;
R₁ is C₁-C₈ alkyl, (—CH₂)ₙ—C₃-C₆ cycloalkyl, wherein n is an integer of from 0 to 3, pyridinyl, —CH₂-pyridinyl, phenyl or benzyl, the rings of the cycloalkyl, pyridinyl, phenyl and benzyl groups being optionally substituted by, from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is H, C₁-C₆ alkyl, C₃-C₆ cycloalkyl, —CH₂—C₃-C₆ cycloalkyl, C₁-C₃ perfluoroalkyl, —CH₂OH or CH₂OAc;
R₃ is H, halogen, C₁-C₆ alkyl, C₁-C₃ perfluoroalkyl, C₁-C₆ alkoxy, C₃-C₆ cycloalkyl, —CH₂—C₃-C₆ cycloalkyl, C₃-C₆ cycloalkenyl, —CH₂—C₃-C₆ cycloalkenyl, —NH₂, or —NO₂;
R₄ is C₃-C₆ cycloalkyl, —CH₂—C₃-C₆ cycloalkyl, C₃-C₆ cycloalkenyl, —CH₂—C₃-C₆ cycloalkenyl, phenyl, benzyl, pyridinyl, or —CH₂-pyridinyl, with the rings of these groups being optionally substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, —NO₂ or (CO)C₁-C₆ alkyl.

Compounds of formula (I) include the following compounds, or pharmaceutically acceptable salt, solvate or ester forms thereof, of formulas (II), (III) and (IV): 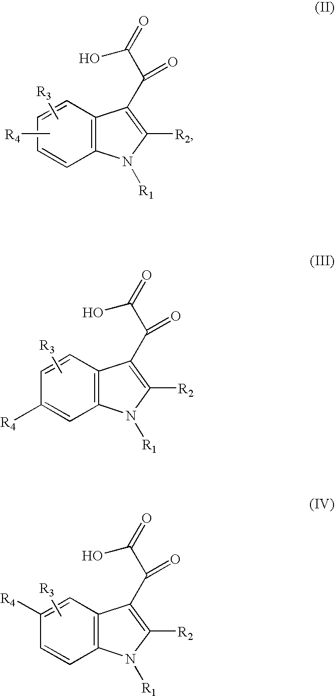
wherein:
R₁, R₂, R₃, and R₄ are as defined previously for formula (I).

Exemplary compounds of formulas I, II, III, and IV include those in which:
R₁ is C₁-C₈ alkyl, (—CH₂)ₙ—C₃-C₆ cycloalkyl, wherein n is an integer of from 0 to 3, pyridinyl, —CH₂-pyridinyl, phenyl or benzyl, the rings of the cycloalkyl, pyridinyl, phenyl and benzyl groups being optionally substituted by, from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is H, C₁-C₆ alkyl, C₃-C₆ cycloalkyl, —CH₂—C₃-C₆ cycloalkyl, C₁-C₃ perfluoroalkyl, —CH₂OH or CH₂OAc;
R₃ is H, halogen, C₁-C₆ alkyl, C₁-C₃ perfluoroalkyl, C₁-C₆ alkoxy, C₃-C₆ cycloalkyl, —CH₂—C₃-C₆ cycloalkyl, C₄-C₆ cycloalkenyl, —CH₂—C₄-C₆ cycloalkenyl, —NH₂, or —NO₂; and
R₄ is phenyl substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, —NO₂ or (CO)C₁-C₆ alkyl; or a pharmaceutically acceptable salt, solvate, or ester form thereof. In certain embodiments, R₄ is at the 4, 5, or 6 position.

Exemplary compounds of formulas I, II, III, and IV include those in which:
R₁ is C₁-C₈ alkyl, (—CH₂)ₙ—C₃-C₆ cycloalkyl, wherein n is an integer of from 0 to 3, or benzyl, the rings of the cycloalkyl, pyridinyl, phenyl and benzyl groups being optionally substituted by, from 1 to 3 groups selected from, halogen, C₁-C₄ alkyl, C₁-C₃ perfluoroalkyl, —O—C₁-C₃ perfluoroalkyl, or C₁-C₃ alkoxy;
R₂ is H, —CH₂OH or CH₂OAc;
R₃ is H;
R₄ is phenyl optionally substituted by from 1 to 3 groups selected from halogen, C₁-C₃ alkyl, C₁-C₃ perfluoroalkyl, —O—C₁-C₃ perfluoroalkyl C₁-C₃ alkoxy or (CO)C₁-C₆ alkyl; or a pharmaceutically acceptable salt, solvate, or ester form thereof. In certain embodiments, R₄ is phenyl substituted by from 1 to 3 groups selected from halogen, C₁-C₃ alkyl, C₁-C₃ perfluoroalkyl, —O—C₁-C₃ perfluoroalkyl C₁-C₃ alkoxy or (CO)C₁-C₆ alkyl.

Exemplary compounds of formulas I, II, III, and IV include those in which:
R₁ is benzyl, the benzyl group being optionally substituted by from 1 to 3 groups selected from halogen, C₁-C₄ alkyl, C₁-C₃ perfluoroalkyl, —O—C₁-C₃ perfluoroalkyl, or C₁-C₃ alkoxy;
R₂ is H;
R₃ is H; and
R₄ is phenyl optionally substituted by from 1 to 3 groups selected from halogen, C₁-C₃ alkyl, C₁-C₃ perfluoroalkyl, —O—C₁-C₃ perfluoroalkyl C₁-C₃ alkoxy or (CO)C₁-C₆ alkyl; or a pharmaceutically acceptable salt, solvate, or ester form thereof. In certain embodiments, R₄ is phenyl substituted by from 1 to 3 groups selected from halogen, C₁-C₃ alkyl, C₁-C₃ perfluoroalkyl, —O—C₁-C₃ perfluoroalkyl C₁-C₃ alkoxy or (CO)C₁-C₆ alkyl.

Compounds of formula (I) include the following compounds, or pharmaceutically acceptable salt, solvate or ester forms thereof, of formula (V) or formula (VI): 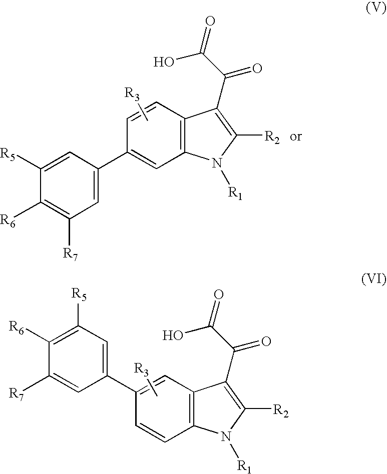
wherein:
R₁ is C₁-C₈ alkyl, C₃-C₆ cycloalkyl, —CH₂—C₃-C₆ cycloalkyl, or benzyl, the rings of the cycloalkyl and benzyl groups being optionally substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is H, C₁-C₆ alkyl, C₃-C₆ cycloalkyl, —CH₂—C₃-C₆ cycloalkyl, or C₁-C₃ perfluoroalkyl;
R₃ is H, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂; and
R₅, R₆ and R₇ are independently H, halogen, C₁-C₃ alkyl, C₁-C₃ perfluoroalkyl, —O—C₁-C₃ perfluoroalkyl, C₁-C₃ alkoxy, —OH, —NH₂, or —NO₂; or a pharmaceutically acceptable salt, solvate, or ester form thereof. In certain embodiments, at least one of R₅, R₆ and R₇ is not H.

Exemplary compounds of formulas V and VI include those in which:
R₁ is benzyl, the benzyl group being optionally substituted by from 1 to 3 groups selected from halogen, C₁-C₄ alkyl, C₁-C₃ perfluoroalkyl, —O—C₁-C₃ perfluoroalkyl, or C₁-C₃ alkoxy;
R₂ is H;
R₃ is H; and
R₅, R₆ and R₇ are independently H, halogen, C₁-C₃ alkyl, C₁-C₃ perfluoroalkyl, —O—C₁-C₃ perfluoroalkyl or C₁-C₃ alkoxy; or a pharmaceutically acceptable salt, solvate, or ester form thereof. In certain embodiments, at least one of R₅, R₆ and R₇ is not H.

Exemplary compounds of formula I include: {1-Methyl-6-[4-(trifluoromethoxy)phenyl]-1H-indol-3-yl}(oxo)acetic acid; {1-Methyl-6-[4-(trifluoromethyl)phenyl]-1H-indol-3-yl}(oxo)acetic acid; {1-Ethyl-6-[4-(trifluoromethoxy)phenyl]-1H-indol-3-yl}(oxo)acetic acid; {1-Ethyl-6-[4-(trifluoromethyl)phenyl]-1H-indol-3-yl}(oxo)acetic acid; {1-Benzyl-6-[4-(trifluoromethoxy)phenyl]-1H-indol-3-yl}(oxo)acetic acid; {1-Benzyl-6-[4-(trifluoromethyl)phenyl]-1H-indol-3-yl}(oxo)acetic acid; {1-[4-(tert-Butyl)benzyl]-6-[4-(trifluoromethyl)phenyl]-1H-indol-3-yl}(oxo)acetic acid; {1-[4-(tert-Butyl)benzyl]-6-[4-(trifluoromethoxy)phenyl]-1H-indol-3-yl}(oxo)acetic acid; {1-Benzyl-5-[4-(trifluoromethyl)phenyl]-1H-indol-3-yl}(oxo)acetic acid; {6-[4-(tert-Butyl)phenyl]-1-methyl-1H-indol-3-yl}(oxo)acetic acid; [5-(4-Acetylphenyl)-1-benzyl-1H-indol-3-yl](oxo)acetic acid; {1-Benzyl-5-[4-(trifluoromethoxy)phenyl]-1H-indol-3-yl}(oxo)acetic acid; {1-Benzyl-4-[4-(trifluoromethyl)phenyl]-1H-indol-3-yl}(oxo)acetic acid; {1-Benzyl-5-[4-(tert-butyl)phenyl]-1H-indol-3-yl}(oxo)acetic acid; [1-Benzyl-5-(3-chloro-4-fluorophenyl)-1H-indol-3-yl](oxo)acetic acid; {1-Benzyl-5-[3,5-bis(trifluoromethyl)phenyl]-1H-indol-3-yl}(oxo)acetic acid; {1-Benzyl-7-[4-(trifluoromethoxy)phenyl]-1H-indol-3-yl}(oxo)acetic acid; [1-Benzyl-7-(3-chloro-4-fluorophenyl)-1H-indol-3-yl](oxo)acetic acid; {1-(4-tert-Butylbenzyl)-5-[4-(trifluoromethoxy)phenyl]-1H-indol-3-yl}(oxo)acetic acid; {1-Benzyl-4-[4-(trifluoromethoxy)phenyl]-1H-indol-3-yl}(oxo)acetic acid; [1-Benzyl-6-(3-chlorophenyl)-1H-indol-3-yl](oxo)acetic acid; {1-Benzyl-5-[3-(trifluoromethoxy)phenyl]-1H-indol-3-yl}(oxo)acetic acid; {1-(4-Methylbenzyl)-5-[4-(trifluoromethoxy)phenyl]-1H-indol-3-yl}(oxo)acetic acid; {1-(4-Fluorobenzyl)-5-[4-(trifluoromethoxy)phenyl]-1H-indol-3-yl}(oxo)acetic acid; [1-Butyl-5-(4-chlorophenyl)-1H-indol-3-yl](oxo)acetic acid; [1-Butyl-5-(3-chlorophenyl)-1H-indol-3-yl](oxo)acetic acid; [1-Butyl-5-(3-methoxyphenyl)-1H-indol-3-yl](oxo)acetic acid; [1-Butyl-5-(4-methoxyphenyl)-1H-indol-3-yl](oxo)acetic acid; {1-Butyl-5-[4-(trifluoromethyl)phenyl]-1H-indol-3-yl}(oxo)acetic acid; [1-(4-tert-Butylbenzyl)-5-(3-methylphenyl)-1H-indol-3-yl](oxo)acetic acid; [1-(4-tert-Butylbenzyl)-5-(3-methoxyphenyl)-1H-indol-3-yl](oxo)acetic acid; [1-(4-tert-Butylbenzyl)-5-(4-tert-butylphenyl)-1H-indol-3-yl](oxo)acetic acid; [1-(4-tert-Butylbenzyl)-5-(3-chlorophenyl)-1H-indol-3-yl](oxo)acetic acid; [1-(4-tert-Butylbenzyl)-5-(4-chlorophenyl)-1H-indol-3-yl](oxo)acetic acid; [1-(4-tert-Butylbenzyl)-5-(2-methylphenyl)-1H-indol-3-yl](oxo)acetic acid; {1-(2-Ethylbutyl)-5-[4-(trifluoromethoxy)phenyl]-1H-indol-3-yl}(oxo)acetic acid; {2-[(Acetyloxy)methyl]-1-(4-methylbenzyl)-5-[4-(trifluoromethoxy)phenyl]-1H-indol-3-yl}(oxo)acetic acid; {2-(Hydroxymethyl)-1-(4-methylbenzyl)-5-[4-(trifluoromethoxy)phenyl]-1H-indol-3-yl}(oxo)acetic acid; {2-[(Acetyloxy)methyl]-1-benzyl-5-[4-(trifluoromethoxy)phenyl]-1H-indol-3-yl}(oxo)acetic acid; {1-Benzyl-2-(hydroxymethyl)-5-[4-(trifluoromethoxy)phenyl]-1H-indol-3-yl}(oxo)acetic acid; [5-(3-Chlorophenyl)-1-cyclopentyl-1H-indol-3-yl]-oxo-acetic acid; [5-(3-chlorophenyl)-1-(cyclobutylmethyl)-1H-indol-3-yl](oxo)acetic acid; [5-(3-chlorophenyl)-1-(3-methylcyclopropyl)-1H-indol-3-yl](oxo)acetic acid; [5-(3-chlorophenyl)-1-(cyclohexylmethyl)-1H-indol-3-yl](oxo)acetic acid; [5-(4-trifluoromethylphenyl)-1-(cyclopentyl)-1H-indol-3-yl](oxo)acetic acid; [5-(4-trifluoromethylphenyl)-1-(cyclobutylmethyl)-1H-indol-3-yl](oxo)acetic acid; [5-(4-trifluoromethylphenyl)-1-(3-methylcyclopentyl)-1H-indol-3-yl](oxo)acetic acid; [5-(4-trifluoromethylphenyl)-1-(cyclohexylmethyl)-1H-indol-3-yl](oxo)acetic acid; [5-(4-trifluoromethylphenyl)-1-(cyclopentylpropyl)-1H-indol-3-yl](oxo)acetic acid; [5-(3-trifluoromethylphenyl)-1-(cyclopentyl)-1H-indol-3-yl](oxo)acetic acid; [5-(3-trifluoromethylphenyl)-1-(cyclobutylmethyl)-1H-indol-3-yl](oxo)acetic acid; [5-(3-trifluoromethylphenyl)-1-(3-methylcyclopentyl)-1H-indol-3-yl](oxo)acetic acid; [5-(3-trifluoromethylphenyl)-1-(cyclohexylmethyl)-1H-indol-3-yl](oxo)acetic acid; [5-(3-trifluoromethylphenyl)-1-(cyclopentylpropyl)-1H-indol-3-yl](oxo)acetic acid; or [5-(4-methoxyphenyl)-1-(cyclohexylmethyl)-1H-indol-3-yl](oxo)acetic acid; or a pharmaceutically acceptable salt, solvate or ester form thereof.

Methods of synthesizing compounds of formula I are provided in U.S. Pat. No. 7,074,817, incorporated herein by reference in its entirety and for all purposes, and are thus not described herein.

In some aspects, this invention describes a method of treating muscle damage, muscle wasting, muscle degeneration, muscle atrophy or reduced rate of muscle repair, wherein said method comprises the administration of an effective amount of a compound of formula (VII), or a pharmaceutically acceptable salt, solvate or ester thereof, to a mammal in need thereof: 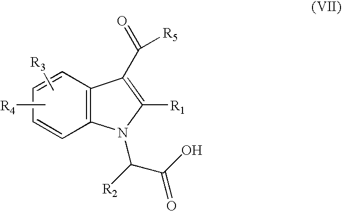
wherein:
R₁ is hydrogen, C₂-C₆ alkyl, C₃-C₆ cycloalkyl, —CH₂—C₃-C₆ cycloalkyl, or C₁-C₃ perfluoroalkyl, wherein the alkyl and cycloalkyl groups are optionally substituted with halogen, —CN, C₁-C₆ alkoxy, —OH, —NH₂, or —NO₂;
R₂ is hydrogen, C₁-C₈ alkyl, C₃-C₆ cycloalkyl, —CH₂—C₃-C₆ cycloalkyl, thienyl, CH₂-thienyl, furanyl, CH₂-furanyl, oxazoyl, CH₂-oxazoyl, phenyl, benzyl, CH₂-naphthyl, wherein the alkyl group and the rings of the cycloalkyl, thienyl, furanyl, oxazoyl, phenyl, benzyl, and napthyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —CO₂R₆, —C(O)NH₂, —S(O)₂CH₃, —OH, —NH₂, or —NO₂;
R₃ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂;
R₄ is C₃-C₈ alkyl, C₃-C₆ alkenyl, C₃-C₆ alkynyl, C₃-C₆ cycloalkyl, —CH₂—C₃-C₆ cycloalkyl, thienyl, furanyl, oxazoyl, phenyl, benzo[b]furan-2-yl, benzo[b]thien-2-yl, benzo[1,3]dioxol-5-yl, naphthyl, wherein the alkyl groups and the rings of the cycloalkyl, thienyl, furanyl, oxazoyl, phenyl, benzofuranyl, benzothienyl, and napthyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, CH₂CO₂H, —C(O)CH₃, —C(O)OR₆, —C(O)NH₂, —S(O)₂CH₃, —OH, —NH₂, or —NO₂;
R₅ is C₁-C₈ alkyl, C₃-C₆ cycloalkyl, —CH₂—C₃-C₆ cycloalkyl, pyridinyl, —CH₂-pyridinyl, thienyl, CH₂-thienyl, furanyl, CH₂-furanyl, oxazoyl, CH₂-oxazoyl, phenyl, benzyl, benzo[b]furan-2-yl, benzo[b]thien-2-yl, benzo[1,3]dioxol-5-yl, naphthyl, CH₂-naphthyl, 9H-fluoren-1-yl, 9H-fluoren-4-yl, 9H-fluoren-9-yl, 9-fluorenone-1-yl, 9-fluorenone-2-yl, 9-fluorenone-4-yl, CH₂-9H-fluoren-9-yl, wherein the alkyl group and the rings of the cycloalkyl, pyridinyl, thienyl, furanyl, oxazoyl, phenyl, benzyl, benzofuranyl, benzothienyl, napthyl, fluorenyl, and fluorenone groups are optionally substituted by from 1 to 3 groups selected from halogen, C₁-C₃ alkyl, C₃-C₆ cycloalkyl, C₁-C₃ perfluoroalkyl, —O—C₁-C₃ perfluoroalkyl, —S—C₁-C₃ perfluoroalkyl, C₁-C₃ alkoxy, phenoxy, —OCHF₂, —CN, —COOH, —CH₂CO₂H, —C(O)CH₃, —CO₂R₆, —C(O)NH₂, —S(O)₂CH₃, —OH, —NH₂, or —NO₂, wherein the phenoxy group are optionally substituted by from 1 to 3 groups selected from halogen, C₁-C₃ alkyl, or C₁-C₃ perfluoroalkyl; and
R₆ is C₁-C₆ alkyl, C₃-C₆ cycloalkyl, —CH₂—C₃-C₆ cycloalkyl, or benzyl; or a pharmaceutically acceptable salt, solvate, or ester form thereof

Exemplary compounds of formula VII include those in which R₁-R₃ and R₅-R₆ are as defined herein for formula VII, and R₄ is thienyl, furanyl, oxazoyl, phenyl, benzo[b]furan-2-yl, benzo[b]thien-2-yl, benzo[1,3]dioxol-5-yl, or naphthyl, wherein the rings of the thienyl, furanyl, oxazoyl, phenyl, benzofuranyl, benzothienyl, and napthyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)OR₆, —C(O)NH₂, —S(O)₂CH₃, —OH, —NH₂, or —NO₂.

Exemplary compounds of formula (VII) include: [3-(4-chlorobenzoyl)-5-(4-chlorophenyl)-1H-indol-1-yl]acetic acid; [3-(Benzo[b]thiophene-2-carbonyl)-5-(4-methylphenyl)-1H-indol-1-yl]-acetic acid; [3-(4-chlorobenzoyl)-5-(4-methylphenyl)-1H-indol-1-yl]-acetic acid; or a pharmaceutically acceptable salt, solvate, or ester form thereof.

Methods of synthesizing compounds of formula I are provided in US Publication No. 20040122070, incorporated herein by reference in its entirety and for all purposes, and are thus not described herein.

In some aspects, this invention describes a method of treating muscle damage, muscle wasting, muscle degeneration, muscle atrophy or reduced rate of muscle repair, wherein said method comprises the administration of an effective amount of a compound of formula (VIII), or a pharmaceutically acceptable salt, solvate or ester thereof, to a mammal in need thereof: 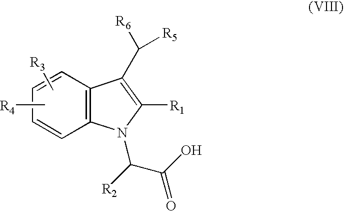
wherein:
R₁ is hydrogen, C₁-C₆ alkyl, C₃-C₆ cycloalkyl, —CH₂—C₃-C₆ cycloalkyl, or C₁-C₃ perfluoroalkyl, wherein the alkyl and cycloalkyl groups are optionally substituted by halogen, —CN, C₁-C₆ alkoxy, —OH, —NH₂, or —NO₂;
R₂ is hydrogen, C₁-C₈ alkyl, C₃-C₆ cycloalkyl, —CH₂—C₃-C₆ cycloalkyl, thienyl, CH₂-thienyl, furanyl, CH₂-furanyl, oxazoyl, CH₂-oxazoyl, phenyl, benzyl, or CH₂-naphthyl; wherein the alkyl group and the rings of the cycloalkyl, thienyl, furanyl, oxazoyl, phenyl, benzyl, and naphthyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)OR₇, —C(O)NH₂, —S(O)₂CH₃, —OH, —NH₂, or —NO₂;
R₃ is hydrogen, halogen, C₁-C₆ alkyl, C₁-C₃ perfluoroalkyl, C₁-C₆ alkoxy, C₃-C₆ cycloalkyl, or —CH₂—C₃-C₆ cycloalkyl;
R₄ is C₃-C₈ alkyl, C₃-C₆ cycloalkyl, —CH₂—C₃-C₆ cycloalkyl, thienyl, CH₂-thienyl, furanyl, oxazoyl, phenyl, benzo[b]furan-2-yl, benzo[b]thien-2-yl, benzo[1,3]dioxol-5-yl, or naphthyl; wherein the alkyl group and the rings of the cycloalkyl, thienyl, furanyl, oxazoyl, phenyl, benzofuranyl, benzothienyl, and napthyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —C(O)CH₃, —C(O)OR₇, —C(O)NH₂, —S(O)₂CH₃, —OH, —NH₂, or —NO₂;
R₅ is C₁-C₈ alkyl, C₃-C₆ cycloalkyl, —CH₂—C₃-C₆ cycloalkyl, pyridinyl, —CH₂-pyridinyl, thienyl, CH₂-thienyl, furanyl, CH₂-furanyl, oxazoyl, CH₂-oxazoyl, phenyl, benzyl, benzo[b]furan-2-yl, benzo[b]thien-2-yl, benzo[1,3]dioxol-5-yl, naphthyl, CH₂-naphyl, 9H-fluoren-1-yl, 9H-fluoren-4-yl, 9H-fluoren-9-yl, 9-fluorenone-1-yl, 9-fluorenone-2-yl, 9-fluorenone-4-yl, or CH₂-9H-fluoren-9-yl; wherein the alkyl group and the rings of the cycloalkyl, pyridinyl, thienyl, furanyl, oxazoyl, phenyl, benzyl, benzofuranyl, benzothienyl, napthyl, fluorenyl, and fluorenone groups are optionally substituted by from 1 to 3 groups selected from halogen, C₁-C₃ alkyl, C₃-C₆ cycloalkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)OR₇, —C(O)NH₂, —S(O)₂CH₃, —OH, —NH₂, —NO₂, or phenoxy, the phenoxy group being further optionally substituted by from 1 to 3 groups selected from halogen, C₁-C₃ alkyl, or C₁-C₃ perfluoroalkyl;
R₆ is hydrogen, C₁-C₈ alkyl, C₃-C₆ cycloalkyl, —CH₂—C₃-C₆ cycloalkyl, pyridyl, thienyl, CH₂-thienyl, furanyl, CH₂-furanyl, oxazoyl, CH₂-oxazoyl, phenyl, benzyl, benzo[b]furan-2-yl, benzo[b]thien-2-yl, benzo[1,3]dioxol-5-yl, CH₂-1-naphthyl, or CH₂-2-naphyl; wherein the alkyl group and the rings of the cycloalkyl, thienyl, furanyl, oxazoyl, phenyl, benzyl, benzofuranyl, benzothienyl, and napthyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)OR₇, —C(O)NH₂, —S(O)₂CH₃, —OH, —NH₂, or —NO₂;
or R₅ and R₆ taken together are C₃-C₆ cycloalkyl, 3-indan-1-yl, 1,2,3,4-tetrahydronaphthalen-1-yl, chroman-4-yl, 4H-chromen-4-yl, thiochroman-4-yl, 9H-fluoren-9-yl, 9,10-dihydroanthracen-9-yl, 9H-xanthen-9-yl, 9H-thioxanthen-9-yl, 6,7,8,9-tetrahydro-5H-benzocyclohepten-5-yl, or 10,11-dihydro-5H-dibenzo[a,d]cyclohepten-5-yl, wherein these groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)OR₇, —C(O)NH₂, —S(O)₂CH₃, —OH, —NH₂, or —NO₂; and
R₇ is C₁-C₆ alkyl, C₃-C₆ cycloalkyl, —CH₂—C₃-C₆ cycloalkyl, or benzyl; or a pharmaceutically acceptable salt, solvate, or ester form thereof

Exemplary compounds of formula VIII include those in which R₁-R₃ and R₅-R₇ are as defined herein for formula VIII, and
R₄ is thienyl, furanyl, oxazoyl, phenyl, benzo[b]furan-2-yl, benzo[b]thien-2-yl, benzo[1,3]dioxol-5-yl, or naphthy; wherein the rings of the thienyl, furanyl, oxazoyl, phenyl, benzofuranyl, benzothienyl, and napthyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, CO₂R₇, —C(O)NH₂, —S(O)₂CH₃, —OH, —NH₂, or —NO₂; or a pharmaceutically acceptable salt, solvate, or ester form thereof.

Compounds of formula (VIII) include the following compounds, or pharmaceutically acceptable salt, solvate or ester forms thereof, of formula (IX): 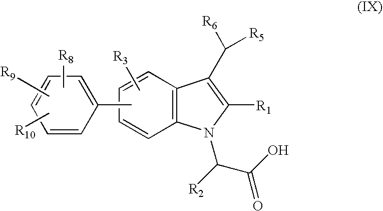
wherein:
R₁ is hydrogen, C₁-C₆ alkyl, C₃-C₆ cycloalkyl, —CH₂—C₃-C₆ cycloalkyl, or C₁-C₃ perfluoroalkyl, wherein the alkyl and cycloalkyl groups are optionally substituted by halogen, —CN, C₁-C₆ alkoxy, —OH, —NH₂, or —NO₂;
R₂ is hydrogen, C₁-C₈ alkyl, C₃-C₆ cycloalkyl, or —CH₂—C₃-C₆ cycloalkyl, wherein the alkyl group and the rings of the cycloalkyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)OR₇, —C(O)NH₂, —S(O)₂CH₃, —OH, —NH₂, or —NO₂;
R₃ is hydrogen, halogen, C₁-C₆ alkyl, C₁-C₃ perfluoroalkyl, C₁-C₆ alkoxy, C₃-C₆ cycloalkyl, or —CH₂—C₃-C₆ cycloalkyl;
R₅ is C₁-C₈ alkyl, C₃-C₆ cycloalkyl, —CH₂—C₃-C₆ cycloalkyl, phenyl, benzyl, naphthyl, or CH₂-naphyl, wherein the alkyl group and the rings of the cycloalkyl, phenyl, and benzyl groups are optionally substituted by from 1 to 3 groups selected from halogen, C₁-C₃ alkyl, C₃-C₆ cycloalkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)OR₇, —C(O)NH₂, —S(O)₂CH₃, —OH, —NH₂, —NO₂, or phenoxy; the phenoxy group being optionally substituted by from 1 to 3 groups selected from halogen, C₁-C₃ alkyl, or C₁-C₃ perfluoroalkyl;
R₆ is hydrogen, C₁-C₈ alkyl, C₃-C₆ cycloalkyl, or —CH₂—C₃-C₆ cycloalkyl, wherein the alkyl group and the rings of the cycloalkyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)NH₂, —S(O)₂CH₃, —OH, —NH₂, or —NO₂;
or R₅ and R₆ taken together are a C₃-C₆ cycloalkyl group optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)NH₂, —S(O)₂CH₃, —OH, —NH₂, or —NO₂;
R₇ is C₁-C₆ alkyl, C₃-C₆ cycloalkyl, —CH₂—C₃-C₆ cycloalkyl, or benzyl; and
R₈, R₉, R₁₀ are each independently hydrogen, halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —C(O)CH₃, —C(O)OR₇, —C(O)NH₂, —S(O)₂CH₃, —OH, —NH₂, or —NO₂; or a pharmaceutically acceptable salt, solvate, or ester form thereof

Compounds of formula (VIII) include the following compounds, or pharmaceutically acceptable salt, solvate or ester forms thereof, of formula (X): 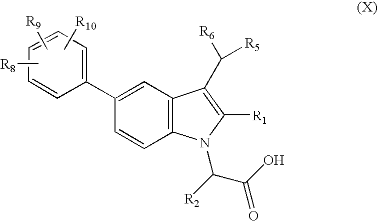
wherein:
R₁ is hydrogen or C₁-C₆ alkyl;
R₂ is hydrogen or C₁-C₃ alkyl, optionally substituted by halogen;
R₅ is C₁-C₈ alkyl, C₃-C₆ cycloalkyl, —CH₂—C₃-C₆ cycloalkyl, phenyl, benzyl, or thienyl, wherein the alkyl group and the rings of the cycloalkyl, phenyl, thienyl and benzyl groups are optionally substituted by from 1 to 3 groups selected from halogen, C₁-C₃ alkyl, C₃-C₆ cycloalkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)NH₂, —S(O)₂CH₃, —OH, —NH₂, or —NO₂;
R₆ is hydrogen or C₁-C₆ alkyl;
R₇ is C₁-C₆ alkyl, C₃-C₆ cycloalkyl, —CH₂—C₃-C₆ cycloalkyl, or benzyl; and
R₈, R₉, R₁₀ are each independently hydrogen, halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —C(O)CH₃, —C(O)NH₂, —S(O)₂CH₃, —OH, —NH₂, or —NO₂; or a pharmaceutically acceptable salt or ester form thereof.

Exemplary compounds of formula VIII, IX, and X include those in which R₅ is C₁-C₈ alkyl, C₃-C₆ cycloalkyl, or —CH₂—C₃-C₆ cycloalkyl, wherein the alkyl group and the rings of the cycloalkyl group are optionally substituted by from 1 to 3 groups selected from halogen, C₁-C₃ alkyl, C₃-C₆ cycloalkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —C(O)OR₇, —C(O)NH₂, —S(O)₂CH₃, —OH, —NH₂, —NO₂, or phenoxy; the phenoxy group being optionally substituted by from 1 to 3 groups selected from halogen, C₁-C₃ alkyl, or C₁-C₃ perfluoroalkyl.

Exemplary compounds of formula (VIII) include: {5-(3-trifluoromethoxyphenyl)-3-[1-(4-trifluoromethylphenyl)-ethyl]-indol-1-yl}-acetic acid; {3-[3,5-bis(trifluoromethyl)benzyl]-5-[4-(trifluoromethoxy)phenyl]-1H-indol-1-yl}acetic)acid; [3-[3,5-bis(trifluoromethyl)benzyl]-5-(2,4-dichlorophenyl)-1H-indol-1-yl]acetic acid; {3-[3,5-bis(trifluoromethyl)benzyl]-5-[3-(trifluoromethyl)phenyl]-1H-indol-1-yl}acetic acid; {5-(3-chlorophenyl)-3-[1-(2-thienyl)ethyl]-1H-indol-1-yl}acetic acid; [3-(1-phenylethyl)-5-(3-trifluoromethyl-phenyl)-indol-1-yl]acetic acid; [3-(1-thiophen-2-yl-ethyl)-5-(3-trifluoromethyl-phenyl)-indol-1-yl]acetic acid; [3-(1-cyclohexyl-ethyl)-5-(3-trifluoromethyl-phenyl)-indol-1-yl]acetic acid; [3-(4-isopropyl-benzyl)-5-(3-trifluoromethyl-phenyl)-indol-1-yl]acetic acid; [5-(2,4-dichloro-phenyl)-3-(1,3-dimethyl-butyl)-indol-1-yl]-acetic acid; [5-(2,4-dichloro-phenyl)-3-(1-phenyl-ethyl)-indol-1-yl]-acetic acid; [3-(1-cyclohexyl-ethyl)-5-(2,4-dichloro-phenyl)-indol-1-yl]-acetic acid; or a pharmaceutically acceptable salt, solvate, or ester form thereof.

Methods of synthesizing compounds of formula VIII are provided in US20060178412, incorporated herein by reference in its entirety and for all purposes, and are thus not described herein.

In some aspects, this invention describes a method of treating muscle damage, muscle wasting, muscle degeneration, muscle atrophy or reduced rate of muscle repair, wherein said method comprises the administration of an effective amount of a compound of formula (XI), or a pharmaceutically acceptable salt, solvate or ester thereof, to a mammal in need thereof: 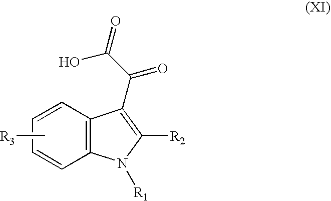
wherein:
R₁ is the moiety: 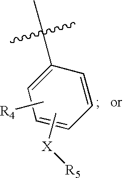
R₁ is C₁-C₈ alkyl, benzo[1,3]dioxo-5yl-methyl, cycloalkylalkyl where the alkyl chain is C₁-C₃, heteroarylalkyl where the alkyl chain is C₁-C₃, arylalkyl where the alkyl chain is C₁-C₃, preferably selected from benzyl, CH₂-1-naphthyl, CH₂-2-naphyl, CH₂CH₂-phenyl, or CH₂CH₂-napthyl, wherein the alkyl, cycloalkyl, heteroaryl, and aryl groups are optionally substituted by from 1 to 3 groups selected from halogen, C₁-C₃ alkyl, C₁-C₃ haloalkyl, C₁-C₃ perfluoroalkyl, C₁-C₃ alkoxy, C₁-C₃ perfluoroalkoxy, C₁-C₃ alkylthio, C₁-C₃ perfluoroalkylthio, —OCHF₂, —CN, —C(O)CH₃, —CO₂R₇, —C(O)NH₂, —S(O)₂CH₃, —OH, —NH₂, or —NO₂;
R₄ is hydrogen, halogen, C₁-C₃ alkyl, C₁-C₃ haloalkyl, C₁-C₃ perfluoroalkyl, C₁-C₃ alkoxy, C₁-C₃ perfluoroalkoxy, C₁-C₃ alkylthio, C₁-C₃ perfluoroalkylthio, —OCHF₂, —CN, —COOH, —CH₂CO₂H, —C(O)CH₃, —CO₂R₇, —C(O)NH₂, —S(O)₂CH₃, —OH, —NH₂, or —NO₂;
X is O, S, or NH;
R₅ is C₁-C₈ alkyl, C₁-C₃ perfluoroalkyl, C₃-C₆ cycloalkyl, —CH₂—C₃-C₆ cycloalkyl, heteroaryl, —CH₂-heteroaryl, phenyl, or arylalkyl where the alkyl chain is C₁-C₈, wherein the rings of the cycloalkyl, heteroaryl, phenyl, and aryl groups are optionally substituted by from 1 to 5 groups selected from halogen, C₁-C₆ alkyl, C₁-C₃ haloalkyl, C₁-C₃ perfluoroalkyl, C₁-C₃ alkoxy, C₁-C₃ perfluoroalkoxy, C₁-C₃ alkylthio, C₁-C₃ perfluoroalkylthio, heteroaryl, —OCHF₂, —CN, —COOH, —CH₂CO₂H, —C(O)CH₃, —CO₂R₇, —C(O)NH₂, —S(O)₂CH₃, —OH, —NH₂, or —NO₂;
R₂ is hydrogen, C₁-C₆ alkyl, —CH₂—C₃-C₆ cycloalkyl, or C₁-C₃ perfluoroalkyl, wherein the alkyl and cycloalkyl groups are optionally substituted by halogen, —CN, C₁-C₆ alkoxy, —COOH, —CH₂CO₂H, —C(O)CH₃, —CO₂R₇, —C(O)NH₂, —S(O)₂CH₃, —OH, —NH₂, or —NO₂;
R₃ is hydrogen, halogen, C₁-C₈ alkyl, C₁-C₈ alkenyl, C₁-C₈ alkynyl, C₃-C₆ cycloalkyl, —CH₂—C₃-C₆ cycloalkyl, heteroaryl, or phenyl, wherein the alkyl, alkenyl, alkynyl, cycloalkyl, heteroaryl, and phenyl groups are optionally substituted by from 1 to 3 groups selected from halogen, C₁-C₃ alkyl, C₁-C₃ haloalkyl, C₁-C₃ perfluoroalkyl, C₁-C₃ alkoxy, C₁-C₃ perfluoroalkoxy, C₁-C₃ alkylthio, C₁-C₃ perfluoroalkylthio, —OCHF₂, —CN, —COOH, —CH₂CO₂H, —C(O)CH₃, —CO₂R₇, —C(O)NH₂, —S(O)₂CH₃, —OH, —NH₂, or —NO₂;
or R₃ is the moiety X—R₆;
R₆ is C₁-C₈ alkyl, C₁-C₈ alkenyl, C₁-C₈ alkynyl, C₃-C₆ cycloalkyl, —CH₂—C₃-C₆ cycloalkyl, heteroaryl, phenyl, aryl-alkyl where the alkyl chain is C₁-C₈, CH₂CH₂-phenyl, or CH₂CH₂-napthyl, wherein the alkyl, alkenyl, alkynyl, cycloalkyl, heteroaryl, and aryl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —C(O)CH₃, —CO₂R₇, —S(O)₂CH₃, —OH, —NH₂, or —NO₂; and
R₇ is C₁-C₆ alkyl, C₃-C₆ cycloalkyl, —CH₂—C₃-C₆ cycloalkyl, or C₁-C₈ aryl-alkyl; or a pharmaceutically acceptable salt, solvate, or ester form thereof.

In certain aspects R₁ is C₁-C₈ alkyl, benzo[1,3]dioxo-5yl-methyl, cycloalkylalkyl where the alkyl chain is C₁-C₃, heteroarylalkyl where the alkyl chain is C₁-C₃, benzyl, CH₂-1-naphthyl, CH₂-2-naphyl, CH₂CH₂-phenyl, or CH₂CH₂-napthyl, wherein the alkyl, cycloalkyl, heteroaryl, benzyl, phenyl, and naphthyl groups are optionally substituted by from 1 to 3 groups selected from halogen, C₁-C₃ alkyl, C₁-C₃ haloalkyl, C₁-C₃ perfluoroalkyl, C₁-C₃ alkoxy, C₁-C₃ perfluoroalkoxy, C₁-C₃ alkylthio, C₁-C₃ perfluoroalkylthio, —OCHF₂, —CN, —C(O)CH₃, —CO₂R₇, —C(O)NH₂, —S(O)₂CH₃, —OH, —NH₂, or —NO₂.

Compounds of formula (XI) include the following compounds, or pharmaceutically acceptable salt, solvate or ester forms thereof, of formula (XII) or (XIII): 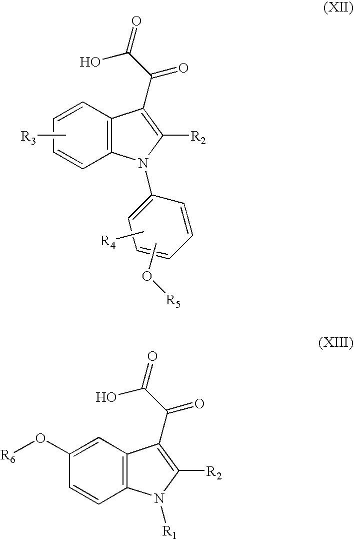
wherein:
R₁ is C₁-C₈ alkyl, benzo[1,3]dioxo-5yl-methyl, cycloalkylalkyl where the alkyl chain is C₁-C₃, heteroarylalkyl where the alkyl chain is C₁-C₃, arylalkyl where the alkyl chain is C₁-C₃, preferably selected from benzyl, CH₂-1-naphthyl, CH₂-2-naphyl, CH₂CH₂-phenyl, or CH₂CH₂-napthyl, wherein the alkyl, cycloalkyl, heteroaryl and aryl groups are optionally substituted by from 1 to 3 groups selected from halogen, C₁-C₃ alkyl, C₁-C₃ perfluoroalkyl, —O—C₁-C₃ perfluoroalkyl, S—C₁-C₃ perfluoroalkyl, C₁-C₃ alkoxy, —OCHF₂, —CN, —COOH, —CH₂CO₂H, —C(O)CH₃, —CO₂R₇, —C(O)NH₂, —S(O)₂CH₃, —OH, —NH₂, or —NO₂;
R₄ is hydrogen, halogen, C₁-C₆ alkyl, C₁-C₃ haloalkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —C(O)CH₃, —CO₂R₇, —S(O)—₂CH₃, —OH, —NH₂, or —NO₂;
R₅ is C₁-C₈ alkyl, C₁-C₃ perfluoroalkyl, —CH₂—C₃-C₆ cycloalkyl, —CH₂-heteroaryl, or aryl-alkyl where the alkyl chain is C₁-C₈, wherein the rings of the cycloalkyl, heteroaryl, and aryl groups are optionally substituted by from 1 to 5 groups selected from halogen, C₁-C₆ alkyl, C₁-C₃ perfluoroalkyl, —O—C₁-C₃ perfluoroalkyl, heteroaryl, S—C₁-C₃ perfluoroalkyl, C₁-C₃ alkoxy, —OCHF₂, —CN, —C(O)CH₃, —CO₂R₇, —S(O)₂CH₃, —OH, —NH₂, or —NO₂;
R₂ is hydrogen, C₁-C₆ alkyl, or C₁-C₃ perfluoroalkyl, wherein the alkyl group is optionally substituted by halogen, —CN, C₁-C₆ alkoxy, —COOH, —CH₂CO₂H, —C(O)CH₃, —CO₂R₇, —C(O)NH₂, —S(O)₂CH₃, —OH, —NH₂, or —NO₂;
R₃ is hydrogen, halogen, C₁-C₈ alkyl, C₁-C₈ alkenyl, C₁-C₈ alkynyl, C₃-C₆ cycloalkyl, —CH₂—C₃-C₆ cycloalkyl, heteroaryl, or phenyl, wherein the alkyl, alkenyl, alkynyl, cycloalkyl, heteroaryl, and phenyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —C(O)CH₃, —CO₂R₇, —S(O)₂CH₃, —OH, —NH₂, or —NO₂;
R₆ is C₁-C₈ alkyl, C₁-C₈ alkenyl, C₁-C₈ alkynyl, C₃-C₆ cycloalkyl, —CH₂—C₃-C₆ cycloalkyl, heteroaryl, phenyl, aryl-alkyl where the alkyl chain is C₁-C₈, CH₂CH₂-phenyl, or CH₂CH₂-napthyl, wherein the alkyl, alkenyl, alkynyl, cycloalkyl, heteroaryl, and aryl groups are optionally substituted by from 1 to 3 groups selected from halogen, C₁-C₃ alkyl, C₁-C₃ perfluoroalkyl, —O—C₁-C₃ perfluoroalkyl, preferably —OCF₃, —S—C₁-C₃ perfluoroalkyl, C₁-C₃ alkoxy, —OCHF₂, —CN, —C(O)CH₃, —CO₂R₇, —S(O)₂CH₃, —OH, —NH₂, or —NO₂; and
R₇ is C₁-C₆ alkyl, C₃-C₆ cycloalkyl, —CH₂—C₃-C₆ cycloalkyl, or aryl-alkyl where the alkyl chain is C₁-C₈; or a pharmaceutically acceptable salt, solvate, or ester form thereof

Exemplary compounds of the formula (XI), (XII), and (XIII) include those in which R₅ is C₁-C₈ alkyl, C₁-C₃ perfluoroalkyl, C₃-C₆ cycloalkyl, —CH₂—C₃-C₆ cycloalkyl, heteroaryl, —CH₂-heteroaryl, phenyl, or arylalkyl where the alkyl chain is C₁-C₈, wherein the rings of the cycloalkyl, heteroaryl, phenyl, and aryl groups are optionally substituted by from 1 to 5 groups selected from halogen, C₁-C₃ alkyl, C₁-C₃ haloalkyl, C₁-C₃ perfluoroalkyl, C₁-C₃ alkoxy, C₁-C₃ perfluoroalkoxy, C₁-C₃ alkylthio, C₁-C₃ perfluoroalkylthio, —OCHF₂, —CN, —COOH, —CH₂CO₂H, —C(O)CH₃, —CO₂R₇, —C(O)NH₂, —S(O)₂CH₃, —OH, —NH₂, or —NO₂;

Exemplary compounds of formula (XI) include: (1-{4-[(4-cyanobenzyl)oxy]phenyl}-1H-indol-3-yl)(oxo)acetic acid; {1-[4-(3-methoxy-benzyloxy)-phenyl]1H-indol-3-yl}-oxo-acetic acid; {1-[4-(3-chloro-benzyloxy)-phenyl]1H-indol-3-yl}-oxo-acetic acid; {1-[4-(4-cyanobenzyloxy)-phenyl]-5-fluoro-1H-indol-3-yl}-oxo-acetic acid; {1-[4-(3,5-dimethoxy-benzyloxy)-phenyl]-5-fluoro-1H-indol-3-yl}-oxo-acetic acid; {1-[4-(3-chloro-benzyloxy)-phenyl]-5-methyl-1H-indol-3-yl}-oxo-acetic acid; {1-[4-(2,4-dichlorobenzyloxy)-phenyl]-5-methyl-1H-indol-3-yl}-oxo-acetic acid; {5-Chloro-1-[3-(4-cyano-benzyloxy)-phenyl]1H-indol-3-yl}-oxo-acetic acid; {5-Chloro-1-[3-(3,5-dimethoxy benzyloxy)-phenyl]1H-indol-3-yl}-oxo-acetic acid; {1-[4-(2,3,5,6-tetrafluoro-4-trifluoromethyl-benzyloxy)-phenyl]1H-indol-3-yl}-oxo-acetic acid; {1-[4-(2,6-dichloro-pyridin-4-ylmethoxy)-phenyl]1H-indol-3-yl}-oxo-acetic acid; [1-(4-{[5-(ethoxycarbonyl)-2-furyl]methoxy}phenyl)-5-fluoro-1H-indol-3-yl](oxo)acetic acid; {1-[4-(2,6-dichloropyridin-4-ylmethoxy)-phenyl]-5-methyl-1H-indol-3-yl}-oxo-acetic acid; {5-Chloro-1-[3-(2,3,5,6-tetrafluoro-4-trifluoromethyl-benzyloxy)-phenyl]1H-indol-3-yl}-oxo-acetic acid; [5-chloro-1-(3-{[5-(ethoxycarbonyl)-2-furyl]methoxy}phenyl)-1H-indol-3-yl](oxo)acetic acid; 5-Chloro-1-[3-(2,6-dichloro-pyridin-4-ylmethoxy)-phenyl]1H-indol-3-yl}-oxo-acetic acid; [1,5-bis-(4-trifluoromethoxy-phenyl)-1H-indol-3-yl]-oxo-acetic acid; [1,5-bis-(4-trifluoromethoxy-phenyl)-1H-indol-3-yl]-oxo-acetic acid; {1-(4-fluorobenzyl)-5-[2-(4-fluorophenyl)ethoxy]-1H-indol-3-yl}(oxo)acetic acid, [1-benzyl-5-(2-chloro-4-trifluoromethyl-phenoxy)-1H-indol-3-yl](oxo)acetic acid; (1-benzyl-5-benzyloxy-1H-indol-3-yl)-oxo-acetic acid; (5-allyloxy-1-cyclobutylmethyl-1H-indol-3-yl)-oxo-acetic acid; (5-allyloxy-1-phenethyl-1H-indol-3-yl)-oxo-acetic acid; (5-allyloxy-1-benzo[1,3]dioxol-5-ylmethyl-1H-indol-3-yl)-oxo-acetic acid; (5-allyloxy-1-[2-(4-methoxyphenyl)-ethyl]-1H-indol-3-yl)-oxo-acetic acid; (5-allyloxy-1-[2-naphthalene-1-yl-ethyl]-1H-indol-3-yl)-oxo-acetic acid; (5-allyloxy-1-[2-(3-trifluoromethylphenyl)-ethyl]-1H-indol-3-yl)-oxo-acetic acid; (5-allyloxy-1-[2-(4-bromophenyl)-ethyl]-1H-indol-3-yl)-oxo-acetic acid; {1-[4-(4-tert-butyl-benzyloxy)-phenyl]-5-methyl-1H-indol-3-yl}-oxo-acetic acid; {1-[4-(4-[1,2,3]thiadiazol-4-yl-benzyloxy)-phenyl]-1H-indol-3-yl}-oxo-acetic acid; {5-Chloro-1-[3-(4-[1,2,3]thiadiazol-4-yl-benzyloxy)-phenyl]1H-indol-3-yl}-oxo-acetic acid; or a pharmaceutically acceptable salt, solvate, or ester form thereof.

Methods of synthesizing compounds of formula (XI) are provided in US20040138283 incorporated herein by reference in its entirety and for all purposes, and are thus not described herein.

In some aspects, this invention describes a method of treating muscle damage, muscle wasting, muscle degeneration, muscle atrophy or reduced rate of muscle repair, wherein said method comprises the administration of an effective amount of a compound of formula (XIV) or (XV), or a pharmaceutically acceptable salt, solvate or ester thereof, to a mammal in need thereof: 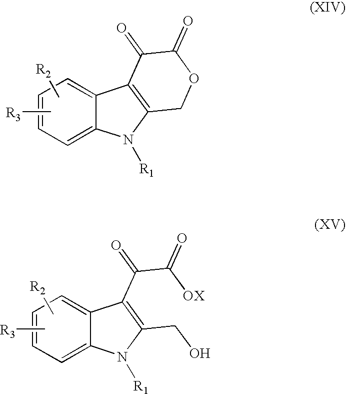
wherein:
X is hydrogen, an alkali metal or a basic amine moiety;
R₁ is hydrogen, C₁-C₈ alkyl, C₃-C₆ cycloalkyl, —CH₂—C₃-C₆ cycloalkyl, pyridinyl, —CH₂-pyridinyl, phenyl or benzyl, wherein the rings of the cycloalkyl, pyridinyl, phenyl and benzyl groups are optionally substituted by from 1 to 3 groups selected from halogen, C₁-C₆ alkyl, C₁-C₆ perfluoroalkyl, —O—C₁-C₆ perfluoroalkyl, C₁-C₆ alkoxy, —OH, —NH₂, or —NO₂;
R₂ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, hydroxy, —NH₂, or —NO₂;
R₃ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, hydroxy, —NH₂, —NO₂, phenyl, benzyl, benzyloxy, pyridinyl, or —CH₂-pyridinyl, wherein the rings of these groups are optionally substituted by from 1 to 3 groups selected from phenyl, halogen, C₁-C₆ alkyl, C₁-C₆ perfluoroalkyl, —O—C₁-C₆ perfluoroalkyl, C₁-C₆ alkoxy, —OH, —NH₂, or —NO₂; or a pharmaceutically acceptable salt, solvate, or ester form thereof

Compounds of formula (XIV) include the following compounds, or pharmaceutically acceptable salt, solvate or ester forms thereof, of formula (XVI) and (XVII): 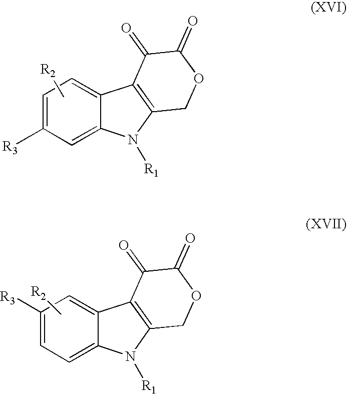
wherein:
R₁ is hydrogen, C₁-C₈ alkyl, C₃-C₆ cycloalkyl, —CH₂—C₃-C₆ cycloalkyl, pyridinyl, —CH₂-pyridinyl, phenyl or benzyl, wherein the rings of the cycloalkyl, pyridinyl, phenyl and benzyl groups are optionally substituted by from 1 to 3 groups selected from halogen, C₁-C₆ alkyl, C₁-C₆ perfluoroalkyl, —O—C₁-C₆ perfluoroalkyl, C₁-C₆ alkoxy, —OH, —NH₂, or —NO₂;
R₂ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂;
R₃ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, hydroxy, —NH₂, —NO₂, phenyl, benzyl, benzyloxy, pyridinyl, or —CH₂-pyridinyl, wherein the rings of these groups are optionally substituted by from 1 to 3 groups selected from halogen, C₁-C₆ alkyl, C₁-C₆ perfluoroalkyl, —O—C₁-C₆ perfluoroalkyl, C₁-C₆ alkoxy, —OH, —NH₂, or —NO₂; or a pharmaceutically acceptable salt, solvate, or ester form thereof

Compounds of formula (XIV) include the following compounds, or pharmaceutically acceptable salt, solvate or ester forms thereof, of formula (XVIII) and (XIX): 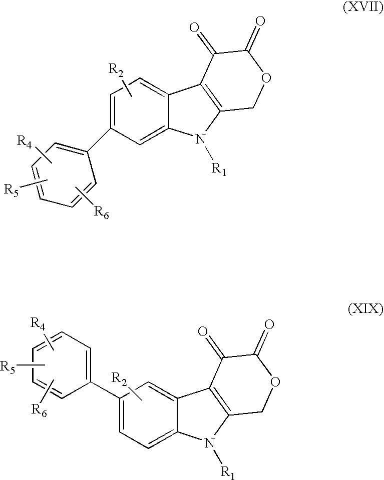
wherein:
R₁ is hydrogen, C₁-C₈ alkyl, preferably C₁-C₆ alkyl, C₃-C₆ cycloalkyl, —CH₂—C₃-C₆ cycloalkyl, or benzyl, wherein the rings of the cycloalkyl and benzyl groups are optionally substituted by from 1 to 3 groups selected from halogen, C₁-C₆ alkyl, C₁-C₆ perfluoroalkyl, —O—C₁-C₆ perfluoroalkyl, C₁-C₆ alkoxy, —OH, —NH₂, or —NO₂;
R₂ is hydrogen, halogen, C₁-C₆ alkyl, C₁-C₃ perfluoroalkyl, preferably —CF₃, C₁-C₆ alkoxy, C₃-C₆ cycloalkyl, —CH₂—C₃-C₆ cycloalkyl, hydroxy, —NH₂, or —NO₂;
R₄, R₅ and R₆ are each independently hydrogen, phenyl, halogen, C₁-C₃ alkyl, C₁-C₃ perfluoroalkyl, —O—C₁-C₃ perfluoroalkyl, C₁-C₃ alkoxy, —OH, —NH₂, or —NO₂; or a pharmaceutically acceptable salt, solvate, or ester form thereof

Exemplary compounds of the formula (XIV), (XV), (XVI), (XVII), (XVIII), and (XIX) include those in which R₁ is C₁-C₈ alkyl, C₃-C₆ cycloalkyl, —CH₂—C₃-C₆ cycloalkyl, pyridinyl, —CH₂-pyridinyl, phenyl or benzyl, wherein the rings of the cycloalkyl, pyridinyl, phenyl and benzyl groups are optionally substituted by from 1 to 3 groups selected from halogen, C₁-C₆ alkyl, C₁-C₆ perfluoroalkyl, —O—C₁-C₆ perfluoroalkyl, C₁-C₆ alkoxy, —OH, —NH₂, or —NO₂.

Exemplary compounds formula (XV) include those wherein the alkali metal is for example, sodium, potassium, lithium, calcium, magnesium, or the like and the basic amine moiety is, for example, amonia, primary amines, secondary amines, tertiary amines, pyridine, aromatic amines, benzyl amines, and the like.

Exemplary compounds of formulas (XIV) and (XV) include: 9-(4-Methylbenzyl)-6-[4-(trifluoromethoxy)phenyl]-1,9-dihydropyrano[3,4-b]indole-3,4-dione; 9-Benzyl-6-[4-(trifluoromethoxy)phenyl]-1,9-dihydropyrano[3,4-b]indole-3,4-dione; 9-(4-Methylbenzyl)-6-(3-Methylphenyl)-1,9-dihydropyrano[3,4-b]indole-3,3-dione; 9-(4-tert-butylbenzyl)-6-(3-Methylphenyl)-1,9-dihydropyrano[3,4-b]indole-3,4-dione; 6-(Benzyloxy)-9-(4-methylbenzyl)-1,9-dihydropyrano[3,4-b]indole-3,4-dione; 6-(Benzyloxy)-1,9-dihydropyrano[3,4-b]indole-3,4-dione; 6-(Benzyloxy)-9-(4-tertbutylbenzyl)-1,9-dihydropyrano[3,4-b]indole-3,4-dione; 9-(4-tertbutybenzyl)-6-hydroxy-1,9-dihydropyrano[3,4-b]indole-3,4-dione; 9-benzyl-6-(4-chlorophenyl)-1,9-dihydropyrano[3,4-b]indole-3,4-dione; [1-benzyl-5-(4-chlorophenyl)-2-(hydroxymethyl)-1H-indole-3-yl](oxo)acetic acid; [1-benzyl-5-(1,1-biphenyl-4-yl)-2-(hydroxymethyl)-1H-indole-3-yl](oxo)acetic acid; 9-benzyl-6-(3-Methylphenyl)-1,9-dihydropyrano[3,4-b]indole-3,4-dione; 9-benzyl-6-(1-1-bi-phenyl-4-yl)-1,9-dihydropyrano[3,4-b]indole-3,4-dione; or a pharmaceutically acceptable salt, solvate, or ester form thereof

Methods of synthesizing compounds of formula (XI) are provided in US20050113436 incorporated herein by reference in its entirety and for all purposes, and are thus not described herein.

In some aspects, this invention describes a method of treating muscle damage, muscle wasting, muscle degeneration, muscle atrophy or reduced rate of muscle repair, wherein said method comprises the administration of an effective amount of a compound of formula (XX), or a pharmaceutically acceptable salt, solvate or ester thereof, to a mammal in need thereof: 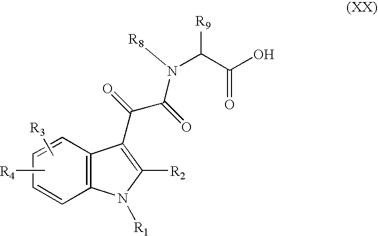
wherein:
R₁ is C₁-C₈ alkyl, C₃-C₆ cycloalkyl, —CH₂—C₃-C₆ cycloalkyl, pyridinyl, —CH₂-pyridinyl, phenyl or benzyl, the rings of the cycloalkyl, pyridinyl, phenyl and benzyl groups are optionally substituted by from 1 to 3 groups selected from the group consisting of halogen, C₁-C₆ alkyl, C₁-C₃ perfluoroalkyl, —O—C₁-C₃ perfluoroalkyl, C₁-C₃ alkoxy, —OH, —NH₂, and —NO₂;
R₂ is hydrogen, C₁-C₆ alkyl, C₃-C₆ cycloalkyl, —CH₂—C₃-C₆ cycloalkyl, or C₁-C₃ perfluoroalkyl;
R₃ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂;
R₄ is phenyl, benzyl, benzyloxy, pyridinyl, or —CH₂-pyridinyl, wherein the rings of these groups are optionally substituted by 1 to 3 groups selected from the group consisting of halogen, C₁-C₃ alkyl, C₁-C₃ perfluoroalkyl, —O—C₁-C₃ perfluoroalkyl, C₁-C₃ alkoxy, —OH, —NH₂, and —NO₂;
R₈ is hydrogen, C₁-C₆ alkyl, C₃-C₆ cycloalkyl, —CH₂—C₃-C₆ cycloalkyl, C₁-C₃ perfluoroalkyl, aryl, substituted aryl, alkyl-aryl, or substituted alkyl-aryl; and
R₉ is hydrogen, C₁-C₆ alkyl, C₃-C₆ branched alkyl, C₁-C₆ hydroxyalkyl, 4-hydroxybenzyl, 3-indolylymethylene, 4-imidazolylmethylene, HSCH₂—, CH₃SCH₂CH₂—, H₂NC(═O)CH₂—, H₂NC(═O)CH₂CH₂—, HO₂CCH₂—, HO₂CCH₂CH₂—, H₂NCH₂CH₂CH₂CH₂—, H₂NC(═NH)NHCH₂CH₂CH₂—, or taken together with R₈, —CH₂CH₂CH₂—;
   or a pharmaceutically acceptable salt, solvate, or ester form thereof.

Exemplary compounds of formula (XX) include those in which R₉ is hydrogen, C₁-C₆ alkyl, C₃-C₆ branched alkyl, 4-hydroxybenzyl, 3-indolylymethylene, 4-imidazolylmethylene, —CH₃SCH₂CH₂—, H₂NC(═O)CH₂—, H₂NC(═O)CH₂CH₂—, HO₂CCH₂—, HO₂CCH₂CH₂—, H₂NCH₂CH₂CH₂CH₂—, H₂NC(═NH)NHCH₂CH₂CH₂—, or taken together with R₈, —CH₂CH₂CH₂—.

Compounds of formula (XX) include the following compounds, or pharmaceutically acceptable salt, solvate or ester forms thereof, of formulas (XXI) and (XXII): 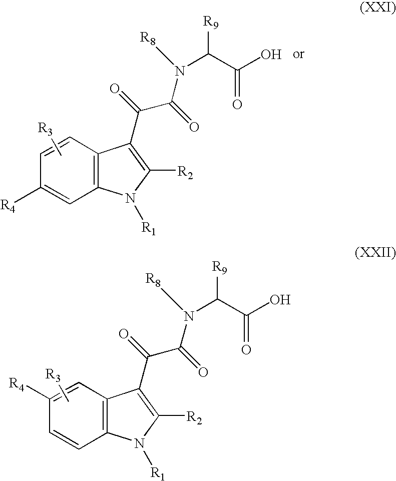
wherein R₁, R₂, R₃, R₄, R₈ and R₉ are as defined herein for a compound of formula (XX), or a pharmaceutically acceptable salt, solvate, or ester form thereof.

Compounds of formula (XX) include the following compounds, or pharmaceutically acceptable salt, solvate or ester forms thereof, of formulas (XXIII) and (XXIV): 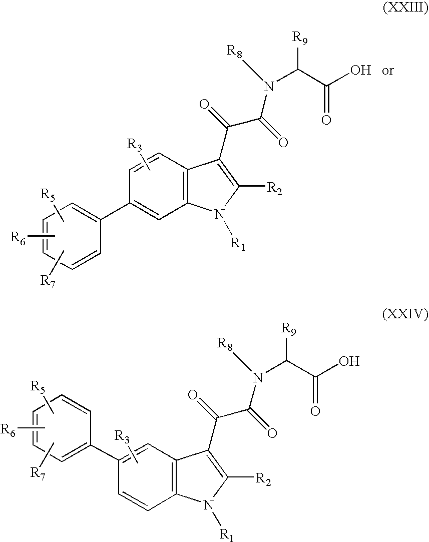
wherein:
R₁ is C₁-C₈ alkyl, C₃-C₆ cycloalkyl, —CH₂—C₃-C₆ cycloalkyl, or benzyl, wherein the rings of the cycloalkyl and benzyl groups are optionally substituted by from 1 to 3 groups selected from halogen, C₁-C₃ alkyl, C₁-C₃ perfluoroalkyl, —O—C₁-C₃ perfluoroalkyl, preferably —O—CF₃, C₁-C₃ alkoxy, —OH, —NH₂, or —NO₂;
R₂ is hydrogen, C₁-C₆ alkyl, C₃-C₆ cycloalkyl, —CH₂—C₃-C₆ cycloalkyl, or C₁-C₃ perfluoroalkyl;
R₃ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂;
R₅, R₆ and R₇ are each independently hydrogen, halogen, C₁-C₃ alkyl, C₁-C₃ perfluoroalkyl, —O—C₁-C₃ perfluoroalkyl, C₁-C₃ alkoxy, —OH, —NH₂, or —NO₂;
R₈ is hydrogen, C₁-C₆ alkyl, C₃-C₆ cycloalkyl, —CH₂—C₃-C₆ cycloalkyl, C₁-C₃ perfluoroalkyl, aryl, substituted aryl, alkyl-aryl, or substituted alkyl-aryl;
R₉ is hydrogen, C₁-C₆ alkyl, C₃-C₆ branched alkyl, C₁-C₆ hydroxyalkyl, 4-hydroxybenzyl, 3-indolylymethylene, 4-imidazolylmethylene, HSCH₂—, CH₃SCH₂CH₂—, H₂NC(═O)CH₂—, H₂NC(═O)CH₂CH₂—, HO₂CCH₂—, HO₂CCH₂CH₂—, H₂NCH₂CH₂CH₂CH₂—, H₂NC(═NH)NHCH₂CH₂CH₂—, or taken together with R₈, —CH₂CH₂CH₂—; or a pharmaceutically acceptable salt or ester form thereof.

Exemplary compounds of formulas (XXIII) and (XXIV) include those in which R₉ is hydrogen, C₁-C₆ alkyl, C₃-C₆ branched alkyl, 4-hydroxybenzyl, 3-indolylymethylene, 4-imidazolylmethylene, CH₃SCH₂CH₂—, H₂NC(═O)CH₂—, H₂NC(═O)CH₂CH₂—, HO₂CCH₂—, HO₂CCH₂CH₂—, H₂NCH₂CH₂CH₂CH₂—, H₂NC(═NH)NHCH₂CH₂CH₂—, or taken together with R₈, —CH₂CH₂CH₂—.

Exemplary compounds of formula (XX) include: {[[1-(4-tert-butylbenzyl)-5-(3-methylphenyl)-1H-indol-3-yl](oxo)acetyl]amino}acetic acid; 2-[(2-{1-Benzyl-5-[4-(trifluoromethoxy)phenyl]-1H-indol-3-yl}-2-oxoacetyl)amino]acetic acid; 2-[(2-{1-Benzyl-5-[3-(trifluoromethoxy)phenyl]-1H-indol-3-yl}-2-oxoacetyl)(methyl)amino]acetic acid; or a pharmaceutically acceptable salt or ester form thereof.

Methods of synthesizing compounds of formula XX are provided in US Publication Number 20040116504 incorporated herein by reference in its entirety and for all purposes, and are thus not described herein.

The present invention provides, inter alia, pharmaceutical compositions useful for the treatment of muscle damage, muscle wasting, muscle degeneration, muscle atrophy or reduced rate of muscle repair, wherein said pharmaceutical compositions comprise a PAI-1 inhibitor. In certain aspects the PAI-1 inhibitors useful for the methods of this invention have a molecular weight of less than 1,000. Exemplary PAI-1 inhibitors include those compounds described herein, for example, the compounds of formulas (I)-(XXIV) or pharmaceutically acceptable salt, solvate, or ester forms thereof.

The present invention provides, inter alia, methods for the treatment of muscle damage, muscle wasting, muscle degeneration, muscle atrophy or reduced rate of muscle repair, wherein said method comprises the administration of a pharmaceutical composition comprising a PAI-1 inhibitor to a mammal in need thereof. In certain aspects the PAI-1 inhibitors useful for the methods of this invention have a molecular weight of less than 1,000. In exemplary embodiments of the present invention, the compounds and compositions of the present invention are used to increase muscle weight, i.e., skeletal muscle weight, in mammals in need thereof, i.e., in mammals having a condition characterized by muscle damage, muscle wasting, muscle degeneration, muscle atrophy or reduced rate of muscle repair.

In some aspects, the PAI-1 inhibitors useful in compositions for the methods of this invention are described in US20060014725, US20050215626, US20050119327, US20050119326, US20050119296, US20050113439, US20050113438, US20050113438, US20050113436, US20050113428, US20050096377, US20050070592, US20050070587, US20050070585, US20050070584, US20040266733, US20040138283, US20040122070, US20040116504, US20040116488, US20030125371, US20030045560, US20030032626, US20030018067, and US20030013732, which are herein incorporated by reference in their entirety.

The present invention provides, inter alia, pharmaceutical compositions useful for the treatment of muscle damage, muscle wasting, muscle degeneration, muscle atrophy or reduced rate of muscle repair, wherein said composition comprises an effective amount of the compounds of the present invention including the compounds of formulas (I)-(XXIV), and at least one pharmaceutically acceptable excipient.

In some embodiments, this invention describes a pharmaceutical composition useful for the treatment of muscle damage, muscle wasting, muscle degeneration, muscle atrophy or reduced rate of muscle repair, wherein said composition comprises an effective amount of formula: {1-Methyl-6-[4-(trifluoromethoxy)phenyl]-1H-indol-3-yl}(oxo)acetic acid; {1-Methyl-6-[4-(trifluoromethyl)phenyl]-1H-indol-3-yl}(oxo)acetic acid; {1-Ethyl-6-[4-(trifluoromethoxy)phenyl]-1H-indol-3-yl}(oxo)acetic acid; {1-Ethyl-6-[4-(trifluoromethyl)phenyl]-1H-indol-3-yl}(oxo)acetic acid; {1-Benzyl-6-[4-(trifluoromethoxy)phenyl]-1H-indol-3-yl}(oxo)acetic acid; {1-Benzyl-6-[4-(trifluoromethyl)phenyl]-1H-indol-3-yl}(oxo)acetic acid; {1-[4-(tert-Butyl)benzyl]-6-[4-(trifluoromethyl)phenyl]-1H-indol-3-yl}(oxo)acetic acid; {1-[4-(tert-Butyl)benzyl]-6-[4-(trifluoromethoxy)phenyl]-1H-indol-3-yl}(oxo)acetic acid; {1-Benzyl-5-[4-(trifluoromethyl)phenyl]-1H-indol-3-yl}(oxo)acetic acid; {6-[4-(tert-Butyl)phenyl]-1-methyl-1H-indol-3-yl}(oxo)acetic acid; [5-(4-Acetylphenyl)-1-benzyl-1H-indol-3-yl](oxo)acetic acid; {1-Benzyl-5-[4-(trifluoromethoxy)phenyl]-1H-indol-3-yl}(oxo)acetic acid; {1-Benzyl-4-[4-(trifluoromethyl)phenyl]-1H-indol-3-yl}(oxo)acetic acid; {1-Benzyl-5-[4-(tert-butyl)phenyl]-1H-indol-3-yl}(oxo)acetic acid; [1-Benzyl-5-(3-chloro-4-fluorophenyl)-1H-indol-3-yl](oxo)acetic acid; {1-Benzyl-5-[3,5-bis(trifluoromethyl)phenyl]-1H-indol-3-yl}(oxo)acetic acid; {1-Benzyl-7-[4-(trifluoromethoxy)phenyl]-1H-indol-3-yl}(oxo)acetic acid; [1-Benzyl-7-(3-chloro-4-fluorophenyl)-1H-indol-3-yl](oxo)acetic acid; {1-(4-tert-Butylbenzyl)-5-[4-(trifluoromethoxy)phenyl]-1H-indol-3-yl}(oxo)acetic acid; {1-Benzyl-4-[4-(trifluoromethoxy)phenyl]-1H-indol-3-yl}(oxo)acetic acid; [1-Benzyl-6-(3-chlorophenyl)-1H-indol-3-yl](oxo)acetic acid; {1-Benzyl-5-[3-(trifluoromethoxy)phenyl]-1H-indol-3-yl}(oxo)acetic acid; {1-(4-Methylbenzyl)-5-[4-(trifluoromethoxy)phenyl]-1H-indol-3-yl}(oxo)acetic acid; {1-(4-Fluorobenzyl)-5-[4-(trifluoromethoxy)phenyl]-1H-indol-3-yl}(oxo)acetic acid; [1-Butyl-5-(4-chlorophenyl)-1H-indol-3-yl](oxo)acetic acid; [1-Butyl-5-(3-chlorophenyl)-1H-indol-3-yl](oxo)acetic acid; [1-Butyl-5-(3-methoxyphenyl)-1H-indol-3-yl](oxo)acetic acid; [1-Butyl-5-(4-methoxyphenyl)-1H-indol-3-yl](oxo)acetic acid; {1-Butyl-5-[4-(trifluoromethyl)phenyl]-1H-indol-3-yl}(oxo)acetic acid; [1-(4-tert-Butylbenzyl)-5-(3-methylphenyl)-1H-indol-3-yl](oxo)acetic acid; [1-(4-tert-Butylbenzyl)-5-(3-methoxyphenyl)-1H-indol-3-yl](oxo)acetic acid; [1-(4-tert-Butylbenzyl)-5-(4-tert-butylphenyl)-1H-indol-3-yl](oxo)acetic acid; [1-(4-tert-Butylbenzyl)-5-(3-chlorophenyl)--1H-indol-3-yl](oxo)acetic acid; [1-(4-tert-Butylbenzyl)-5-(4-chlorophenyl)-1H-indol-3-yl](oxo)acetic acid; [1-(4-tert-Butylbenzyl)-5-(2-methylphenyl)- 1H-indol-3-yl](oxo)acetic acid; {1-(2-Ethylbutyl)-5-[4-(trifluoromethoxy)phenyl]-1H-indol-3-yl}(oxo)acetic acid; {2-[(Acetyloxy)methyl]-1-(4-methylbenzyl)-5-[4-(trifluoromethoxy)phenyl]-1H-indol-3-yl}(oxo)acetic acid; {2-(Hydroxymethyl)-1-(4-methylbenzyl)-5-[4-(trifluoromethoxy)phenyl]-1H-indol-3-yl}(oxo)acetic acid; {2-[(Acetyloxy)methyl]-1-benzyl-5-[4-(trifluoromethoxy)phenyl]-1H-indol-3-yl}(oxo)acetic acid; {1-Benzyl-2-(hydroxymethyl)-5-[4-(trifluoromethoxy)phenyl]-1H-indol-3-yl}(oxo)acetic acid; [5-(3-Chlorophenyl)-1-cyclopentyl-1H-indol-3-yl]-oxo-acetic acid; [5-(3-chlorophenyl)-1-(cyclobutylmethyl)-1H-indol-3-yl](oxo)acetic acid; [5-(3-chlorophenyl)-1-(3-methylcyclopropyl)-1H-indol-3-yl](oxo)acetic acid; [5-(3-chlorophenyl)-1-(cyclohexylmethyl)-1H-indol-3-yl](oxo)acetic acid; [5-(4-trifluoromethylphenyl)-1-(cyclopentyl)-1H-indol-3-yl](oxo)acetic acid; [5-(4-trifluoromethylphenyl)-1-(cyclobutylmethyl)-1H-indol-3-yl](oxo)acetic acid; [5-(4-trifluoromethylphenyl)-1-(3-methylcyclopentyl)-1H-indol-3-yl](oxo)acetic acid; [5-(4-trifluoromethylphenyl)-1-(cyclohexylmethyl)-1H-indol-3-yl](oxo)acetic acid; [5-(4-trifluoromethylphenyl)-1-(cyclopentylpropyl)-1H-indol-3-yl](oxo)acetic acid; [5-(3-trifluoromethylphenyl)-1-(cyclopentyl)-1H-indol-3-yl](oxo)acetic acid; [5-(3-trifluoromethylphenyl)-1-(cyclobutylmethyl)-1H-indol-3-yl](oxo)acetic acid; [5-(3-trifluoromethylphenyl)-1-(3-methylcyclopentyl)-1H-indol-3-yl](oxo)acetic acid; [5-(3-trifluoromethylphenyl)-1-(cyclohexylmethyl)-1H-indol-3-yl](oxo)acetic acid; [5-(3-trifluoromethylphenyl)-1-(cyclopentylpropyl)-1H-indol-3-yl](oxo)acetic acid; or [5-(4-methoxyphenyl)-1-(cyclohexylmethyl)-1H-indol-3-yl](oxo)acetic acid; [3-(4-chlorobenzoyl)-5-(4-chlorophenyl)-1H-indol-1-yl]acetic acid; [3-(Benzo[b]thiophene-2-carbonyl)-5-(4-methylphenyl)-1H-indol-1-yl]-acetic acid; [3-(4-chlorobenzoyl)-5-(4-methylphenyl)-1H-indol-1-yl]-acetic acid; {5-(3-trifluoromethoxyphenyl)-3-[1-(4-trifluoromethylphenyl)-ethyl]-indol-1-yl}-acetic acid; {3-[3,5-bis(trifluoromethyl)benzyl]-5-[4-(trifluoromethoxy)phenyl]-1H-indol-1-yl}acetic)acid; [3-[3,5-bis(trifluoromethyl)benzyl]-5-(2,4-dichlorophenyl)-1H-indol-1-yl]acetic acid; {3-[3,5-bis(trifluoromethyl)benzyl]-5-[3-(trifluoromethyl)phenyl]-1H-indol-1-yl}acetic acid; {5-(3-chlorophenyl)-3-[1-(2-thienyl)ethyl]-1H-indol-1-yl}acetic acid; [3-(1-phenylethyl)-5-(3-trifluoromethyl-phenyl)-indol-1-yl]acetic acid; [3-(1-thiophen-2-yl-ethyl)-5-(3-trifluoromethyl-phenyl)-indol-1-yl]acetic acid; [3-(1-cyclohexyl-ethyl)-5-(3-trifluoromethyl-phenyl)-indol-1-yl]acetic acid; [3-(4-isopropyl-benzyl)-5-(3-trifluoromethyl-phenyl)-indol-1-yl]acetic acid; [5-(2,4-dichloro-phenyl)-3-(1,3-dimethyl-butyl)-indol-1-yl]-acetic acid; [5-(2,4-dichloro-phenyl)-3-(1-phenyl-ethyl)-indol-1-yl]-acetic acid; [3-(1-cyclohexyl-ethyl)-5-(2,4-dichloro-phenyl)-indol-1-yl]-acetic acid; (1-{4-[(4-cyanobenzyl)oxy]phenyl}-1H-indol-3-yl)(oxo)acetic acid; {1-[4-(3-methoxy-benzyloxy)-phenyl]1H-indol-3-yl}-oxo-acetic acid; {1-[4-(3-chloro-benzyloxy)-phenyl]1H-indol-3-yl}-oxo-acetic acid; {1-[4-(4-cyanobenzyloxy)-phenyl]-5-fluoro-1H-indol-3-yl}-oxo-acetic acid; {1-[4-(3,5-dimethoxy-benzyloxy)-phenyl]-5-fluoro-1H-indol-3-yl}-oxo-acetic acid; {1-[4-(3-chloro-benzyloxy)-phenyl]-5-methyl-1H-indol-3-yl}-oxo-acetic acid; {1-[4-(2,4-dichlorobenzyloxy)-phenyl]-5-methyl-1H-indol-3-yl}-oxo-acetic acid; {5-Chloro-1-[3-(4-cyano-benzyloxy)-phenyl]1H-indol-3-yl}-oxo-acetic acid; {5-Chloro-1-[3-(3,5-dimethoxy benzyloxy)-phenyl]1H-indol-3-yl}-oxo-acetic acid; {1-[4-(2,3,5,6-tetrafluoro-4-trifluoromethyl-benzyloxy)-phenyl]1H-indol-3-yl}-oxo-acetic acid; {1-[4-(2,6-dichloro-pyridin-4-ylmethoxy)-phenyl]1H-indol-3-yl}-oxo-acetic acid; [1-(4-{[5-(ethoxycarbonyl)-2-furyl]methoxy}phenyl)-5-fluoro-1H-indol-3-yl](oxo)acetic acid; {1-[4-(2,6-dichloropyridin-4-ylmethoxy)-phenyl]-5-methyl-1H-indol-3-yl}-oxo-acetic acid; {5-Chloro-1-[3-(2,3,5,6-tetrafluoro-4-trifluoromethyl-benzyloxy)-phenyl]1H-indol-3-yl}-oxo-acetic acid; [5-chloro-1-(3-{[5-(ethoxycarbonyl)-2-furyl]methoxy}phenyl)-1H-indol-3-yl](oxo)acetic acid; 5-Chloro-1-[3-(2,6-dichloro-pyridin-4-ylmethoxy)-phenyl]1H-indol-3-yl}-oxo-acetic acid; [1,5-bis-(4-trifluoromethoxy-phenyl)-1H-indol-3-yl]-oxo-acetic acid; [1,5-bis-(4-trifluoromethoxy-phenyl)-1H-indol-3-yl]-oxo-acetic acid; {1-(4-fluorobenzyl)-5-[2-(4-fluorophenyl)ethoxy]-1H-indol-3-yl}(oxo)acetic acid, [1-benzyl-5-(2-chloro-4-trifluoromethyl-phenoxy)-1H-indol-3-yl](oxo)acetic acid; (1-benzyl-5-benzyloxy-1H-indol-3-yl)-oxo-acetic acid; (5-allyloxy-1-cyclobutylmethyl-1H-indol-3-yl)-oxo-acetic acid; (5-allyloxy-1-phenethyl-1H-indol-3-yl)-oxo-acetic acid; (5-allyloxy-1-benzo[1,3]dioxol-5-ylmethyl-1H-indol-3-yl)-oxo-acetic acid; (5-allyloxy-1-[2-(4-methoxyphenyl)-ethyl]-1H-indol-3-yl)-oxo-acetic acid; (5-allyloxy-1-[2-naphthalene-1-yl-ethyl]-1H-indol-3-yl)-oxo-acetic acid; (5-allyloxy-1-[2-(3-trifluoromethylphenyl)-ethyl]-1H-indol-3-yl)-oxo-acetic acid; (5-allyloxy-1-[2-(4-bromophenyl)-ethyl]-1H-indol-3-yl)-oxo-acetic acid; {1-[4-(4-tert-butyl-benzyloxy)-phenyl]-5-methyl-1H-indol-3-yl}-oxo-acetic acid; {1-[4-(4-[1,2,3]thiadiazol-4-yl-benzyloxy)-phenyl]-1H-indol-3-yl}-oxo-acetic acid; {5-Chloro-1-[3-(4-[1,2,3]thiadiazol-4-yl-benzyloxy)-phenyl]1H-indol-3-yl}-oxo-acetic acid; 9-(4-Methylbenzyl)-6-[4-(trifluoromethoxy)phenyl]-1,9-dihydropyrano[3,4-b]indole-3,4-dione; 9-Benzyl-6-[4-(trifluoromethoxy)phenyl]-1,9-dihydropyrano[3,4-b]indole-3,4-dione; 9-(4-Methylbenzyl)-6-(3-Methylphenyl)-1,9-dihydropyrano[3,4-b]indole-3,3-dione; 9-(4-tert-butylbenzyl)-6-(3-Methylphenyl)-1,9-dihydropyrano[3,4-b]indole-3,4-dione; 6-(Benzyloxy)-9-(4-methylbenzyl)-1,9-dihydropyrano[3,4-b]indole-3,4-dione; 6-(Benzyloxy)-1,9-dihydropyrano[3,4-b]indole-3,4-dione; 6-(Benzyloxy)-9-(4-tertbutylbenzyl)-1,9-dihydropyrano[3,4-b]indole-3,4-dione; 9-(4-tertbutybenzyl)-6-hydroxy-1,9-dihydropyrano[3,4-b]indole-3,4-dione; 9-benzyl-6-(4-chlorophenyl)-1,9-dihydropyrano[3,4-b]indole-3,4-dione; [1-benzyl-5-(4-chlorophenyl)-2-(hydroxymethyl)-1H-indole-3-yl](oxo)acetic acid; [1-benzyl-5-(1,1-biphenyl-4-yl)-2-(hydroxymethyl)-1H-indole-3-yl](oxo)acetic acid; 9-benzyl-6-(3-Methylphenyl)-1,9-dihydropyrano[3,4-b]indole-3,4-dione; 9-benzyl-6-(1-1-bi-phenyl-4-yl)-1,9-dihydropyrano[3,4-b]indole-3,4-dione; {[[1-(4-tert-butylbenzyl)-5-(3-methylphenyl)-1H-indol-3-yl](oxo)acetyl]amino}acetic acid; 2-[(2-{1-Benzyl-5-[4-(trifluoromethoxy)phenyl]-1H-indol-3-yl}-2-oxoacetyl)amino]acetic acid; 2-[(2-{1-Benzyl-5-[3-(trifluoromethoxy)phenyl]-1H-indol-3-yl}-2-oxoacetyl)(methyl)amino]acetic acid; or a pharmaceutically acceptable salt, solvate or ester thereof, and at least one pharmaceutically acceptable excipient.

In some embodiments of this invention, the compositions of this invention are in the form of a tablet or capsule.

In some embodiments, this invention describes a pharmaceutical composition useful for the treatment of muscle wasting, muscle degeneration, muscular atrophy, or reduced rate of muscle repair, wherein said muscle wasting, muscle degeneration, muscular atrophy, or reduced rate of muscle repair is caused by or associated with muscular dystrophy.

In some embodiments, this invention describes a pharmaceutical composition useful for the treatment of muscle wasting, muscle degeneration, muscular atrophy, or reduced rate of muscle repair caused by or associated with muscular dystrophy of the type Duchenne's, Becker's, distal, ocular, Emery-Dreifuss, facioscapulohumeral, Fukuyama congenital, limb-girdle, myotonic, oculopharyngeal or severe childhood autosomal recessive.

Using the methods of the present invention, one or more compounds or pharmaceutical compositions of the present invention can be administered concomitantly with other known therapies to treat a subject suffering muscle wasting, muscle degeneration, muscular atrophy, or reduced rate of muscle repair. “Concomitant administration” of a known therapy with a pharmaceutical composition of the present invention means administration of the drug and the pharmaceutical composition at such time that both the known drug and the composition of the present invention will have a therapeutic effect. Such concomitant administration can involve concurrent (i.e. at the same time), prior, or subsequent administration of the known therapy with respect to the administration of a compound of the present invention. A person of ordinary skill in the art, would have no difficulty determining the appropriate timing, sequence and dosages of administration for particular drugs and compositions of the present invention. For example, in some embodiments, the compounds of this invention can be used in combination with an agent that decreases the efficacy of endogenous myostatin for the treatment of muscular dystrophy.

In some embodiments, the compounds of this invention can be used in combination with a myostatin antibody for the treatment of muscular dystrophy.

In certain embodiments, the compounds of this invention can be used in combination with prothrombolytic and fibrinolytic agents, which include, but are not limited to tissue plasminogen activator, streptokinase, and activase for the treatment of muscular dystrophy.

In some embodiments of this invention, said method of treating muscular dystrophy further comprises the administration of at least one anabolic agent, including, for example, an anabolic androgen.

In some embodiments of this invention, said method of treating muscular dystrophy further comprises the administration of a glucocorticoid.

The preferred salt forms of the compounds herein include but are not limited to sodium salts, and potassium salts. Other useful salt forms of these compounds include those formed with pharmaceutically acceptable inorganic and organic bases known in the art. Salt forms prepared using inorganic bases include hydroxides, carbonates or bicarbonates of the therapeutically acceptable alkali metals or alkaline earth metals, such as sodium potassium, magnesium, calcium and the like. Acceptable organic bases include amines, such as benzylzmine, mono-, di- and trialkylamines, preferably those having alkyl groups of from 1 to 6 carbon atoms, more preferably 1 to 3 carbon atoms, such as methylamine, dimethylamine, trimethylamine, ethylamine, diethylamine, triethylamine, mono-, di-, and triethanolamine. Also useful are alkylene diamines containing up to 6 carbon atoms, such as hexamethylenediamine; cyclic saturated or unsaturated bases containing up to 6 carbon atoms, including pyrrolidine, peperidine, morpholine, piperazine and their N-alkyl and N-hydroxyalkyl derivatives, such as N-methyl-morpholine and N-(2-hydroxyethyl)-piperidine, or pyridine. Quaternary salts can also be formed, such as tetralkyl forms, such as tetramethyl forms, alkyl-alkanol forms, such as methyl-triethanol or trimethyl-monoethanol forms, and cyclic ammonium salt forms, such as N-methylpyridinium, N-methyl-N-(2-hydroxyethyl)-morpholinium, N,N-di-methylmorpholinium, N-methyl-N-(2-hydroxyethyl)-morpholinium, or N,N-dimethyl-piperidinium salt forms. These salt forms can be prepared using the acidic compound(s) of the formulas described herein and procedures known in the art.

The compounds used in the methods of this invention can contain one or more asymmetric centers, which can thus give rise to optical isomers (enantiomers) and diastereomers. The present invention includes such optical isomers (enantiomers) and diastereomers (geometric isomers); as well as the racemic and resolved, enantiomerically pure R and S stereoisomers; as well as other mixtures of the R and S stereoisomers and pharmaceutically acceptable salts thereof. The use of these compounds is intended to cover the racemic mixture or either of the chiral enantiomers.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

One skilled in the art will also recognize that it is possible for tautomers to exist for the compounds of the present invention. The present invention includes all such tautomers even though not shown in the formulas herein.

Compounds of the invention can also include all isotopes of atoms occurring in the intermediates or final compounds. Isotopes include those atoms having the same atomic number but different mass numbers. For example, isotopes of hydrogen include tritium and deuterium.

Ester forms of the compounds of this invention include straight chain alkyl esters having from 1 to 6 carbon atoms or branched chain alkyl groups containing 3 or 6 carbon atoms, including methyl, ethyl, propyl, butyl, 2-methylpropyl and 1,1-dimethylethyl esters. Other esters useful with this invention include those of the formula —COOR₈ wherein R₈ is selected from the formulae: 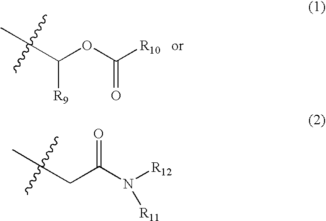
wherein R₉, R₁₀, R₁₁, R₁₂ are independently selected from hydrogen, alkyl of from 1 to 10 carbon atoms, aryl of 6 to 14 carbon atoms, arylalkyl of from 6 to 14 carbon atoms wherein the aryl ring is bound by an alkyl chain of from 1 to 6 carbon atoms; heteroaryl or alkylheteroaryl wherein the heteroaryl ring is bound by an alkyl chain of from 1 to 6 carbon atoms.

Among the preferred ester forms of the compounds herein include but not limited to C₁-C₆ alkyl esters, C₃-C₆ branched alkyl esters, benzyl esters, and the like.

As used herein, “aryl”, employed alone or in combination with other terms, refers to an unsaturated aromatic carbocyclic group of from 6 to 14 carbon atoms having a single ring (e.g., phenyl) or multiple condensed (fused) rings (e.g., naphthyl or anthryl). Preferred aryl groups include phenyl, naphthyl and the like. As used herein, “heteroaryl”, employed alone or in combination with other terms, refers to a monocyclic or bicyclic aromatic group of from about 5 to about 14 carbon atoms and 1 to about 4 heteroatoms selected from oxygen, nitrogen and sulfur within at least one ring (if there is more than one ring). Such heteroaryl groups can have a single ring, such as pyridyl, pyrrolyl or furyl groups, or multiple condensed rings, such as indolyl, indolizinyl, benzofuranyl or benzothienyl groups. Preferred heteroaryls include pyridyl, pyrrolyl and furyl.

Unless otherwise limited by the definition for the aryl or heteroaryl groups herein, such groups can optionally be substituted with from 1 to 5 substituents selected from the group consisting of acyloxy, hydroxy, acyl, alkyl of 1 to 6 carbon atoms, alkoxy of 1 to 6 carbon atoms, alkenyl of 2 to 6 carbon atoms, alkynyl of 2 to 6 carbon atoms, substituted alkyl of 1 to 6 carbon atoms, substituted alkoxy of 1 to 6 carbon atoms, substituted alkenyl of 2 to 6 carbon atoms, substituted alkynyl of 2 to 6 carbon atoms, amino, amino substituted by one or two alkyl groups of from 1 to 6 carbon atoms, aminoacyl, acylamino, azido, cyano, halo, nitro, thioalkoxy of from 1 to 6 carbon atoms, substituted thioalkoxy of from 1 to 6 carbon atoms, and trihalomethyl. Substituents on the alkyl, alkenyl, alkynyl, thioalkoxy and alkoxy groups mentioned above include halogens, CN, OH, and amino groups. Preferred substituents on the aryl groups herein include alkyl, alkoxy, halo, cyano, nitro, trihalomethyl, and thioalkoxy.

The term “alkyl”, employed alone or in combination with other terms, refers to a saturated hydrocarbon group that can be straight-chain or branched. In some embodiments, the alkyl group contains within the range of carbons specified and in some embodiments where not otherwise specified, the alkyl group contains 1 to 10 carbon atoms, preferably 1 to 6 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like.

The term “alkenyl”, as used herein, whether used alone or as part of another group, refers to an aliphatic hydrocarbon chain and includes, but is not limited to, straight and branched chains having 2 to about 10 carbon atoms (unless explicitly specified otherwise) and containing at least one double bond. Preferably, the alkenyl moiety has 1 or 2 double bonds. Preferably, the alkenyl moiety has about 2 to about 7 carbon atoms. Such alkenyl moieties can exist in the E or Z conformations and the compounds of this invention include both conformations.

The term “alkynyl”, as used herein, whether used alone or as part of another group, refers to an aliphatic hydrocarbon chain and includes, but is not limited to, straight and branched chains having 2 to about 10 carbon atoms (unless explicitly specified otherwise) and containing at least one triple bond. Preferably, the alkynyl moiety has about 2 to about 7 carbon atoms. In certain embodiments, the alkynyl can contain more than one triple bond and, in such cases, the alkynyl group must contain at least four carbon atoms.

The term “perfluoroalkyl”, as used herein, whether used alone or as part of another group, refers to a saturated aliphatic hydrocarbon having 1 to 6 carbon atoms and two or more fluorine atoms and includes, but is not limited to, straight or branched chains, such as —CF₃, —CH₂CF₃, —CF₂CF₃ and —CH(CF₃)₂.

The term “halogen” or “halo” refers to chlorine, bromine, fluorine, and iodine.

As used herein, the term “cycloalkyl”, employed alone or in combination with other terms, refers to a non-aromatic cyclic hydrocarbon moiety. Cycloalkyl groups can be characterized as having a range of carbon atoms as specified and one or more backbone carbon atoms of the cycloalkyl group can be double-bonded to an oxygen atom. Cycloalkyl groups have about 3 to about 20 carbon atoms, preferably 3 to about 6 carbon atoms. Exemplary cycloalkyl groups include cyclopropyl, cyclobutyl, cyclopentyl, cyclohexanone and cyclohexyl.

As used herein, the term “cycloalkenyl”, refers to a non-aromatic cyclic hydrocarbon moiety containing 1 or more double bonds within the backbone structure of the cycloalkene moiety. Cycloalkenyl groups can be characterized as having a range of carbon atoms as specified and one or more backbone carbon atoms of the cycloalkenyl group can be double-bonded to an oxygen atom.

The term “acyl”, employed alone or in combination with other terms, is defined herein as, unless otherwise stated, either an alkyl, arylalkyl, heteroarylalkyl, (C2-C10) straight chain, or (C4-C11) branched-chain monovalent hydrocarbon moiety; wherein the carbon atom, covalently linked to the defined chemical structure, is oxidized to the carbonyl oxidation state. Such hydrocarbon moieties may be mono or polyunsaturated, and may exist in the E or Z configurations. Examples of acyl moieties include, but are not limited to, chemical groups such as acetyl, propionyl, butyryl, 3,3-dimethylbutyryl, trifluoroacetyl, pivaloyl, hexanoyl, hexenoyl, decanoyl, benzoyl, nicotinyl, isonicotinyl, and homologs, isomers, and the like.

Exemplary compounds of this invention are active as PAI-1 inhibitors. In inhibiting PAI-1, the compounds can effectively increase the amount of plasminogen, plasmin and thus enhance fibrinolysis. While not wishing to be bound by theory, it is believed that the compounds of this invention prevent muscle wasting and facilitate muscle damage repair via inhibition of the plasminogen activator inhibitor 1. In so doing, increased levels of plasminogen are available in turn generating increased levels of plasmin. Such increased levels of plasmin can increase muscle healing processes via clearance of fibrin clots thus allowing cell migration to the muscle injury site and a corresponding increased rate of muscle repair. Exemplary compounds of this invention have broad applicability to conditions associated with muscle damage, wasting, degeneration, atrophy or reduced rate of repair. Such conditions can come about as a result of other conditions, for example, diabetes, hyperglycemia, motor neuron diseases, carpal tunnel syndrome, chronic infection, tuberculosis, Addison's disease, adult spinal muscular atrophy, anorexia nervosa, dermatomyositis, inclusion body myositis, incontinentia pigmenti, intercoastal neuralgia, juvenile rheumatoid arthritis, legg-calve-perthes disease, multifocal motor neuropathy, nephritic syndrome, osteogenesis imperfecta, post-polio syndrome, spinal muscular atrophy, nerve injury, neuropathy, diabetic neuropathy, alcoholic neuropathy, and muscular dystrophy.

Exemplary compounds of this invention are useful for the treatment of muscle wasting that occurs from immobility and bed rest. Exemplary compounds of this invention are useful in treating damage to muscle tissue, wherein such damage may be normal damage or traumatic damage. Normal damage can occur through routine physical exercise and movement. Traumatic damage may occur where muscles are torn, stretched or otherwise harmed in a violent or sudden fashion.

Muscular dystrophy, as referred to herein, refers to the various forms of muscular dystrophy including Duchenne's, Becker's, distal, congenital, ocular, distal, Emery-Dreifuss, facioscapulohumeral, Fukuyama congenital, limb-girdle, myotonic, oculopharyngeal and severe childhood autosomal recessive.

Methods for the treatment, inhibition, prevention or prophylaxis in a mammal of each of the conditions or maladies listed as well as any and all associated with muscular dystrophy are part of the present invention. Each method comprises administering to a mammal in need thereof a pharmaceutically or therapeutically effective amount of a compound of this invention, or a pharmaceutically acceptable salt or ester or solvate form thereof. The term “treating” refers to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters. Accordingly, the term “treating” includes the administration of the compounds or agents of the present invention to prevent or delay, to alleviate, or to arrest or inhibit development of the symptoms or conditions associated with the disease. The term “therapeutic effect” refers to the reduction, elimination, or prevention of the disease, symptoms of the disease, or side effects of the disease in the subject.

For purposes of this invention, the term “pharmaceutically acceptable excipient” includes any pharmaceutically acceptable substance beside the drug substance (a compound of one of the formulae of this invention) including diluents, fillers and bulking agents, binders and adhesives, propellants, disintegrants, lubricants and glidants, colors, flavors, coating agents, polishing agents, fragrances, sweetening agents, polymers, and waxes.

In some embodiments of this invention, said muscle damage, muscle wasting, muscle degeneration, muscle atrophy or reduced rate of muscle repair is caused by or associated with diabetes, hyperglycemia, motor neuron diseases, carpal tunnel syndrome, chronic infection, tuberculosis, Addison's disease, adult spinal muscular atrophy, anorexia nervosa, dermatomyositis, inclusion body myositis, incontinentia pigmenti, intercoastal neuralgia, juvenile rheumatoid arthritis, legg-calve-perthes disease, multifocal motor neuropathy, nephritic syndrome, osteogenesis imperfecta, post-polio syndrome, spinal muscular atrophy, nerve injury, neuropathy, diabetic neuropathy, or alcoholic neuropathy.

In some embodiments of this invention, said muscle damage is associated with normal muscle exertion or exercise.

In some embodiments of this invention, said muscle damage is associated with traumatic injury to muscle.

In some embodiments of this invention, said muscle wasting, muscle degeneration, muscular atrophy, or reduced rate of muscle repair is caused by or associated with muscular dystrophy.

In some embodiments of this invention, said muscular dystrophy is Duchenne's, Becker's, distal, ocular, Emery-Dreifuss, facioscapulohumeral, Fukuyama congenital, limb-girdle, myotonic, oculopharyngeal or severe childhood autosomal recessive.

In some embodiments of this invention, said muscular dystrophy is Duchenne's.

This invention provides pharmaceutical compositions for use in the methods of this invention comprising a pharmaceutically or therapeutically effective amount of a compound of this invention, or a pharmaceutically acceptable salt or ester or solvate form thereof, either alone or in combination with one or more pharmaceutically acceptable carriers or excipients (i.e. pharmaceutically acceptable materials with no significant pharmacological effects on their own). It will be understood that a pharmaceutically or therapeutically effective amount of a compound herein refers to an amount of the compound in question which will sufficiently inhibit the serine protease inhibitor PAI-1 in the mammal in need thereof to a sufficient extent to provide a desirable improvement in the condition in question or provide sufficient inhibition of the serine protease inhibitor PAI-1 to prevent, inhibit or limit the onset of the physiological basis for the malady or condition in question.

The precise dosage to be employed depends upon several factors including the host, whether in veterinary medicine or human medicine, the nature and severity of the condition being treated, the mode of administration and the particular active substance employed. The compounds can be administered by any conventional route, in particular enterally, preferably orally in the form of tablets or capsules. Administered compounds can be in the free form or pharmaceutically acceptable salt form as appropriate, for use as a pharmaceutical, particularly for use in the muscle conditions. These measures will slow the rate of progress of the disease state and assist the body in reversing the process direction in a natural manner.

Any suitable carrier known to the art can be used to prepare the pharmaceutical compositions. In such a composition, the carrier can be a solid, liquid or mixture of a solid and a liquid. Solid compositions include powders, tablets and capsules. A solid carrier can be one or more substances which can also act as a flavoring agent, lubricant, solubilizer, suspending agent, binder, or tablet disintegrant. In powders, the carrier is a finely divided solid, which is in admixture with the finely divided active ingredient. In tablets, the active ingredient is mixed with a carrier having the necessary binding properties in suitable proportions and compacted in the shape and size desired. Suitable solid carriers are magnesium carbonate, magnesium stearate, talc, sugar, lactose, pectin, dextrin, starch, gelatin, tragacanth, methyl cellulose, hydroxymethyl cellulose, sodium carboxymethyl cellulose, a low melting wax, cocoa butter, and the like. Encapsulating materials can also be employed with the compounds of this invention, and the term “composition” is intended to include the active ingredient in combination with an encapsulating material as a formulation, with or without other carriers. Cachets can also be used in the delivery of the medicaments of this invention.

Sterile liquid compositions include solutions, suspensions, emulsions, syrups and elixirs. The compounds of this invention can be dissolved or suspended in the pharmaceutically acceptable carrier, such as sterile water, sterile organic solvent or a mixture of both. Preferably the liquid carrier is one suitable for parental injection. Where the compounds are sufficiently soluble they can be dissolved directly in normal saline with or without the use of suitable organic solvents, such as propylene glycol or polyethylene glycol. If desired, dispersions of the finely divided compounds can be made-up in aqueous starch or sodium carboxymethyl cellulose solution, or in a suitable oil, such as arachis oil. Liquid pharmaceutical compositions, which are sterile solutions or suspensions, can be utilized by intramuscular, intraperitoneal or subcutaneous injection. In many instances a liquid composition form can be used instead of the preferred solid oral method of administration.

It is preferred to prepare unit dosage forms of the compounds for standard administration regimens. In this way, the composition can be subdivided readily into smaller doses at the physician's direction. For example, unit dosages can be made up in packeted powders, vials or ampoules and preferably in capsule or tablet form. The active compound present in these unit dosage forms of the composition can be present in an amount of from about one gram to about fifteen grams or more, for single or multiple daily administration, according to the particular need of the patient. The daily dose of active compound will vary depending upon the route of administration, the size, age and sex of the patient, the severity of the disease state, and the response to the therapy as traced by blood analysis and the patients recovery rate. By initiating the treatment regimen with a minimal daily dose of about one gram, the blood levels of PAI-1 and the patients symptomatic relief analysis can be used to determine whether a larger dose is indicated. Based upon the data presented below, the projected daily dose for both human and veterinary use will be from about 5 to about 200 milligrams/kilogram per day, and more usually, from about 10 to about 50 milligrams/kilogram per day.

The ability of the compounds of this invention to speed muscle recovery can be examined in various animal models. One model useful for the demonstration of activity is the mouse model disclosed in Am. J. Physiol Cell Physiol. 289: C217-C223, 2005, herein incorporated by reference in its entirety. Briefly, in this model, C57BL/6 (wild-type) mice are anesthethized and the preinjury in situ maximal isometric force of the EDL muscle measured (Am. J. Physiol. Cell Physiol. 286: C713-C722, 2004, herein incorporated by reference in its entirety). Cardiotoxin (Calbiochem, San Diego Calif.) is injected into the EDL in three locations. Subsequent to the injections, the skin is closed and the procedure repeated in the contralateral muscle. After recovery, the mice are subjected to the muscle force evaluation at predetermined intervals. One group of animals serve as the control group and one group of animals is treated with a compound as described in this invention. The compound can be dosed orally, IP or subcutaneously as desired. Multiple treated groups can be used in order to assess dose response relationships if desired. Exemplary compounds of the methods described herein demonstrate the ability to speed a return to normal muscle force pre-injury versus those in the control group. Additional evidence for the compounds' salutary effects can be ascertained by, for example, morphological examination of the damaged muscle tissue including staining of cryosections from treated and control animals and comparing damaged to undamaged area.

Compounds of this invention can also be evaluated in mdx mice (see, for example, Ann Neurol 2002; 52:832-836, herein incorporated by reference in its entirety). Briefly, mdx mice mouse contains a nonsense mutation in the gene for dystrophin resulting in the absence of the protein from the muscle fiber necrosis and the sarcolemma. Groups of mdx mice representing a control group and groups of animals treated with compounds of this invention are compared over a predetermined time period. In particular, at predetermined time periods, animals muscle weights and grip strength are compared (see again Ann Neurol 2002; 52:832-836). After sacrificing of animals at predetermined time points, muscle weight, muscle fiber diameter, muscle histopathology measurements can be taken and hydroxyproline content of the animals diaphragms can be measured and compared between the treated and control groups. Preferred compounds useful for the methods of this invention are those that positively affect one or more of the muscle and/or strength parameters.

Exemplary compounds of formula (I) provided herein can be prepared by the methods disclosed in US2003/0125371 which is herein incorporated by reference in its entirety. The general schemes (1 and 2) are detailed below:

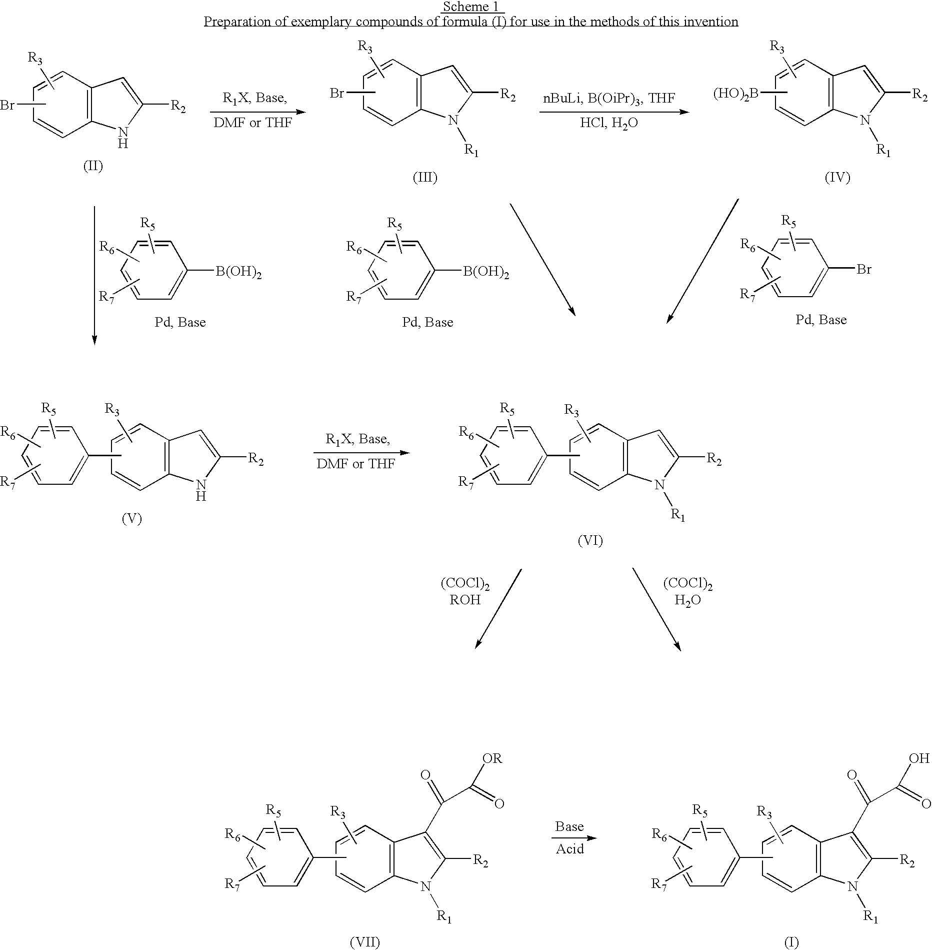

Exemplary compounds of the present invention can be readily prepared according to the methods of scheme 1 or 2 using readily available starting materials, reagents and conventional synthetic procedures. It is also possible to make use of variants of these process steps, which in themselves are known to and well within the preparatory skill of the medicinal chemist. In the reaction schemes outlined above, R₁, R₂, R₃, R₄ and R₅ are selected from the groups as defined previously, or groups readily convertible thereto.

The bromo-indoles (II) were either commercially available or were prepared following known literature procedures (Ayer et. al., Tetrahedron Letters, 48 (14) 2919-2924, 1992; Rapoport et. al., JOC, 51, 5106-5110, 1986, herein incorporated by reference in its entirety).

In the method of Scheme 1, the bromo-indoles (II) can be reacted with alkyl halides or aryl-alkyl halides using a base such as sodium hydride in DMF or THF giving the N-substituted bromo-indoles (III). The N-substituted bromo-indoles (III) can be converted to the corresponding boronic acids (IV) by treating III in THF with nBuLi, followed by triisopropyl-borate and subsequent quenching with aqueous acid. Boronic acids (IV) can then be subjected to palladium catalyzed cross-coupling with various substituted aryl-halides affording the aryl-indoles (VI). Alternatively, N-substituted bromo-indoles (III) can be subjected to the palladium catalyzed cross-coupling with various substituted aryl-boronic acids to afford the aryl-indoles (VI). Furthermore, reaction of bromo-indoles (II) with various substituted aryl-boronic acids under the palladium catalyzed cross-coupling conditions can afford the aryl-indoles (V). Alkylation of (V) with alkyl-halides or aryl-alkyl-halides under basic conditions as described above can afford the N-substituted aryl-indoles (VI). Reaction of (VI) with oxalyl chloride in methylene chloride followed by quenching with water can afford the desired ketoacids (I), which can be purified by crystallization. Alternatively (VI) can be reacted with oxalyl chloride in methylene chloride followed by quenching with alcohol to afford the keto-esters (VII). The ketoesters (VII) can be purified by either crystallization or chromatography. Conversion of the ketoesters (VII) to the corresponding ketoacids (I) can be accomplished by saponification of the ester followed by neutralization with an acid such as hydrochloric acid.

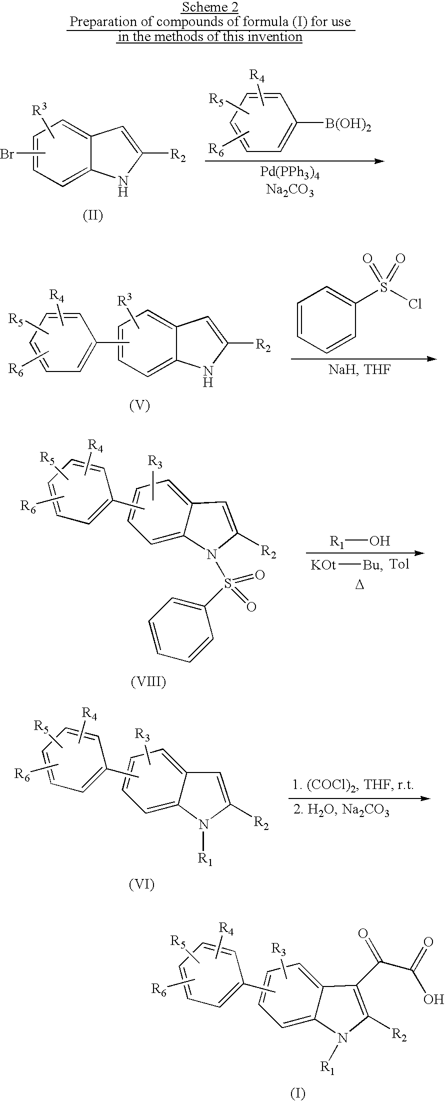

In the method of Scheme 2, an indole (II), substituted on the benzene ring with bromide, iodine, or triflate, is coupled with an aryl boronic acid in the presence of a palladium catalyst, such as Pd(PPh₃)₄, a base, such as Na₂CO₃ or NaHCO₃, in a solvent, such as water, methanol or ethanol, or in a mixed co-solvent system comprising two or more of the aforesaid solvents, at 50-110° C. Boronic acid derivatives of benzene, furan, thiophene, benz[b]thiophene and naphthalene are described in the literature and many are presently commercially available. The resulting aryl indole (V) can be sulfonylated on nitrogen using phenylsulfonyl chloride or toluenesulfonyl chloride in the presence of a base, such as NaH or KOt-Bu, in an inert solvent, such as THF or DMF. The resulting 5-aryl-1H-arylsulfonyl indole (VIII) is reacted with alcohols in the presence of base, such as NaH or KOt-Bu, in an inert solvent, preferably toluene or DMF, at 80-200° C., to give 5-aryl-1H-alkyl indoles (VI). Reaction with oxalyl chloride, neat or in an inert solvent, affords the indol-3-yl glyoxylic chloride. Quenching the reaction with water affords the desired 5-aryl-1H-alkyl indol-3-yl glyoxylic acids (I).

Selected Preparations

Exemplary compounds of formula (I) provided herein can be prepared by the methods disclosed in U.S. Pat. No. 7,074,817, which is herein incorporated by reference in its entirety. Seven of the examples from that disclosure are enclosed herein for illustrative procedures.

### EXAMPLE 1

### {1-Methyl-6-[4-(trifluoromethoxy)phenyl]-1H-indol-3-yl}(oxo)acetic acid

Step 1

### 6-(4-Trifluoromethoxyphenyl)-1H-indole

The mixture of 6-bromo-1H-indole (1.22 g, 6.22 mmol), 4-trifluoromethoxyphenyl boronic acid (1.41 g, 6.84 mmol), tetrakis(triphenylphosphine)palladium (0.213 g, 0.184 mmol) and sodium carbonate (2.64 g, 24.9 mmoles) in water (12.5 mL), ethanol (4 mL), and toluene (25 mL) was heated at reflux for 1.5 hours then cooled to room temperature. The mixture was then evaporated to dryness and the residue was partitioned in methylene chloride and water. The organic phase was washed with water, brine, dried over anhydrous magnesium sulfate and evaporated to dryness. The residue was purified by flash chromatography using 10-30% chloroform in hexane as an eluant. The title compound was obtained as a white solid (0.874 g, 51%), mp: 165-166° C. ¹HNMR (300 MHz, DMSO-d6): δ11.25 (s, 1H), 7.8 (d, 2H, J=7.0 Hz), 7.65 (d, 2H, J=7.0 Hz), 7.4-7.5 (m, 3H), 7.3 (d, 1H, J=8.8 Hz), and 6.45 ppm (s, 1H).

Step 2

### 6-(4-Trifluoromethoxyphenyl)-1-methyl-1H-indole

To a solution of 6-(4-trifluoromethoxyphenyl)-1H-indole (0.853, 3.08 mmoles) in dry THF (10 mL) was added sodium hydride (60% dispersion on mineral oil, 0.47 g, 12.3 mmol), portionwise. The reaction mixture was stirred under nitrogen for 30 minutes and, then cooled in ice bath. A solution of iodomethane (0.38 mL, 6.1 mmole) in dry THF (10 mL) was added and the reaction mixture was stirred for 1 hour at room temperature. The mixture was then poured into excess water, acidified with 2N hydrochloric acid and extracted with ethyl acetate. The organic phase was washed with brine, dried over anhydrous magnesium sulfate and evaporated to dryness. The residue was purified by flash chromatography (Biotage apparatus) using 0.5% t-butyl methyl ether in hexane as an eluant. The title compound was obtained as a cream-colored solid that was vacuum dried at 66° C. (0.623 g, 70%). ¹HNMR (300 MHz, DMSO-d₆): δ7.85 (d, 2H, J=8.3 Hz), 7.75 (s, 1H), 7.65 (d, 1H, J=8.3 Hz), 7.45 (d, 2H, J=8.3 Hz), 7.3-7.4 (m, 2H), 6.45 (s, 1H), and 3.85 ppm (s, 3H).

Step 3

### Methyl 2-[6-(4-trifluoromethoxyphenyl)-1-methyl-1H-indol-3-yl]-2-oxoacetate

To a solution of 6-(4-trifluoromethoxyphenyl)-1-methyl-1H-indole (0.304 g, 1.04 mmol) in dry THF (5 mL) under nitrogen at 0° C. was added oxalyl chloride (0.11 ml, 1.2 mmol). The reaction mixture was stirred at room temperature for 2 hour. The mixture was cooled in an ice bath. Methanol (1 mL) was added. The reaction mixture was stirred at room temperature for 1 hour then poured into excess sodium bicarbonate solution and extracted with ethyl acetate. The organic phase was washed with water and brine, dried over anhydrous magnesium sulfate, and evaporated to dryness. The residue was purified by flash chromatography (Biotage apparatus) using 20-50% ethyl acetate in hexane as an eluant. The title compound was obtained as a cream-colored solid (0.196 g, 50%), mp: 152-153° C. Mass spectrum (+APCI, [M+H]⁺), m/z 378; ¹HNMR (400 MHz, DMSO-d₆): δ8.55 (s, 1H), 8.2 (d, 1H, J=8.3 Hz), 7.95 (t, 1H, J=0.77 Hz), 7.90-7.95 (m, 2H), 7.65 (dd, 1H, J=8.3 Hz and 1.5 Hz), 7.45 (d, 2H, J=8.6 Hz), 4.0 (s, 3H), and 3.9 ppm (m, 3H). Elemental Analysis for C₁₉H₁₄F₃NO₄: Calculated: C, 60.48; H, 3.74; N, 3.71. Found: C, 60.60; H, 3.86; N, 3.60.

Step 4

### {1-Methyl-6-[4-(trifluoromethoxy)phenyl]-1H-indol-3-yl}(oxo)acetic acid

The mixture of methyl 2-[6-(4-trifluoromethoxyphenyl)-1-methyl-1H-indol-3-yl]-2-oxoacetate (0.120 g, 0.318 mmol), and sodium hydroxide (1N, 1 mL, 1.0 mmol) in methanol (10 mL), was stirred at room temperature for 2.5 hours. The mixture was poured into excess water and acidified with 1N hydrochloric acid. The mixture was extracted with ethyl acetate. The organic phase was washed with water, brine and dried over anhydrous magnesium sulfate. The organic phase was evaporated to dryness and dried under vacuum at 55° C. for 12 hours to yield the title compound as a yellow solid (0.0686 g, 59.1%), mp: 233-235° C. (dec). Mass spectrum (+APCI, [M+H]⁺), m/z 364; ¹HNMR (400 MHz, DMSO-d₆): δ13.8-14.0 (br s, 1H), 8.55 (s, 1H), 8.25 (d, 1H, J=8.3 Hz), 7.95 (d, 1H, J=1.1 Hz), 7.85-7.95 (m, 2H), 7.65 (dd, 1H, J=8.2 and 1.6 Hz), 7.45 (dd, 2H, J=8.8 Hz and 0.88 Hz), and 4.0 ppm (s, 3H). Elemental Analysis for C₁₉H₁₄F₃NO₄: Calculated: C, 59.51; H, 3.33; N, 3.86. Found: C, 59.39; H, 3.38; N, 3.71.

### EXAMPLE 2

### {1-Benzyl-6-[4-(trifluoromethoxy)phenyl]-1H-indol-3-yl}(oxo)acetic acid

Step 1

### 1-Benzyl-6-bromo-1H-indole

A solution of 6-bromoindole (5.0 g, 26 mmol) in dry DMF (45 mL) was cooled in an ice bath. Sodium hydride (2.2 g of 60% dispersion in oil, 55 mmol) was added. After stirring for 30 minutes under nitrogen at room temperature, the reaction mixture was cooled in an ice bath, and benzyl bromide (6.1 mL, 51 mmol) was added. After stirring for one hour at room temperature, the reaction mixture was poured into excess water, acidified with 2N hydrochloric acid and extracted with ethyl acetate. The organic phase was then washed with water and brine, then dried over anhydrous magnesium sulfate and evaporated to dryness. Purification of the residue by flash chromatography using hexane as an eluant and drying for 30 minutes at 60° C. yielded 1-benzyl-6-bromo-1H-indole (5.83 g, 80%) as a waxy solid, mp: 85-88° C. Mass spectrum (+ESI, [M+H]⁺), m/z 286; ¹HNMR (500 MHz, DMSO-d₆): δ7.7 (d, 1H, J=0.61 Hz), 7.50-7.55 (m, 2H), 7.30 (t, 2H, J=7.3 Hz), 7.2-7.25 (m, 1H), 7.2 (d, 2H, J=8.4 Hz), 7.15 (dd, 1H, J=8.4 Hz and 1.7 Hz), 6.5 (dd, 1H, J=3.1 Hz and 0.77 Hz), and 5.45 ppm (s, 2H).

Step 2

### 1-Benzyl-6-(4-trifluoromethoxylphenyl)-1H-indole

The mixture of 1-benzyl-6-bromo-1H-indole (0.272 g, 0.950 mmol), 4-trifuoromethoxybenzeneboronic acid (0.217 g, 1.05 mmol), tetrakis(triphenylphosphine) palladium (0.0340 g, 0.0294 mmol) and sodium carbonate (0.405 g, 3.82 mmol) in water (1.9 mL), ethanol (1 mL) and toluene (5 mL) was heated at reflux for 5 hours. The mixture was cooled to room temperature and evaporated to dryness. The residue was partitioned in methylene chloride and water. The organic phase was washed with brine, dried over anhydrous magnesium sulfate and evaporated to dryness. The residue was purified by flash chromatography using hexane as an eluant affording the title compound as thick yellow oil. ¹HNMR (200 MHz, DMSO-d₆): δ7.8 (d, 3H, J=7.7 Hz), 7.65 (d, 2H, J=7.7 Hz), 7.55 (d, 2H, J=2.0 Hz), 7.2-7.5 (m, 5H), 6.55 (d, 1H, J=1.5 Hz), and 5.5 ppm (s, 2H).

Step 3

### Ethyl 2-[1-benzyl-6-(4-trifluoromethoxylphenyl)-1H-indol-3-yl]-2-oxoacetate

Following the procedure described in Step 3 of Example 1, ethyl 2-[1-benzyl-6-(4-trifluoromethoxyphenyl)-1H-indol-3-yl]-2-oxoacetate was prepared from 1-benzyl-6-(4-trifluoromethoxyphenyl)-1H-indole (1.20 g, 3.27 mmol), oxalyl chloride (0.85 mL, 10 mmol) in THF (20 mL), and ethanol (5 mL). Purification by flash chromatography (Biotage apparatus) using 5-10% ethyl acetate in hexane as an eluant yielded the title compound as a yellow gum (0.128 g, 84%). ¹HNMR (300 MHz, DMSO-d₆): δ8.7 (s, 1H), 8.25 (d, 1H, J=7.7 Hz), 8.0 (s, 1H), 7.8 (d, 2H, J=7.7 Hz), 7.65 (dd, 1H, J=7.7 Hz and 0.77 Hz), 7.45 (d, 2H, J=7.7 Hz), 7.25-7.4 (m, 5H), 5.65 (s, 2H), 4.35 (q, 2H, J=7.2 Hz), and 1.35 ppm (t, 3H, J=7.2 Hz).

Step 4

### {1-Benzyl-6-[4-(trifluoromethoxy)phenyl]-1H-indol-3-yl}(oxo)acetic acid

A mixture of ethyl 2-[1-benzyl-6-(4-trifluoromethoxyphenyl)-1H-indol-3-yl]-2-oxoacetate (1.27 g, 2.72 mmol), potassium hydroxide (0.539 g, 9.61 mmol) in THF (12 mL) and water (12 mL) was stirred at room temperature for 3.5 hours. The mixture was poured into excess water, acidified with 2N hydrochloric acid, and extracted with ethyl acetate. The organic phase was washed with water and brine, dried over anhydrous magnesium sulfate, and evaporated to dryness. The residue was dried under vacuum at 92° C. for 15 hours to yield the title compound (0.985 g, 82%) as a yellow solid. A sample crystallized from isopropanol gave a solid, mp: 202-204° C. (dec.). Mass spectrum (+APCI, [M+H]⁺), m/z 440; ¹HNMR (400 MHz, DMSO-d₆): δ13.8-14.2 (br s, 1H), 8.7 (s, 1H), 8.25 (d, 1H, J=8.3 Hz), 7.95 (t, 1H, J=0.73 Hz), 7.8-7.85 (m, 2H), 7.65 (dd, 1H, J=8.3 Hz and 1.5 Hz), 7.45 (d, 2H, J=8.8 Hz), 7.25-7.4 (m, 5H), and 5.65 ppm (s, 2H). Elemental Analysis for C₂₄H₁₆F₃NO₄: Calculated: C, 65.61; H, 3.67; N, 3.19. Found: C, 65.59; H, 3.54; N, 3.18.

### EXAMPLE 3

### [5-(4-Acetylphenyl)-1-benzyl-1H-indol-3-yl](oxo)acetic acid

Step 1

### 1-[4-(1-Benzyl-1H-indol-5-yl)phenyl]-1-ethanone

A mixture of 1-benzyl-5-bromo-1H-indole (1.00 g, 3.49 mmol), 4-acetylphenylboronic acid (0.692 g, 4.22 mmol), [1,1′-bis(diphenylphosphino)ferrocene]dichloropalladium (II) complex with dichloromethane (1:1) (0.0581 g, 0.0711 mmol), potassium carbonate (0.725 g, 5.25 mmol) in dioxane (35 mL) and water (3.5 mL) was heated at 65-70° C. for 3 hours. The reaction mixture was evaporated to dryness and partitioned in ethyl acetate and 2N hydrochloric acid. The organic phase was washed with water and brine, dried over anhydrous magnesium sulfate and evaporated to dryness. The residue was purified by flash chromatography using (1-10% ethyl acetate in hexane and 10-15% chloroform in hexane) to yield 1-[4-(1-benzyl-1H-indol-5-yl)phenyl]-1-ethanone as a buff-colored solid (0.262 g, 23%), mp: 134-135° C. 1HNMR (300 MHz, DMSO-d₆): δ7.95-8.1 (m, 3H), 7.85 (d, 2H, J=7.7 Hz), 7.5-7.65 (m, 3H), 7.2-7.4 (m, 5H), 6.6 (d, 1H, J=2.5 Hz), 5.5 (s, 2H), and 2.6 ppm (s, 3H).

Step 2

### Ethyl 2-[5-(4-acetylphenyl)-1-benzyl-1H-indol-3-yl]-2-oxoacetate

Ethyl 2-[5-(4-acetylphenyl)-1-benzyl-1H-indol-3-yl]-2-oxoacetate was prepared from 1-[4-(1-benzyl-1H-indol-5-yl)phenyl]-1-ethanone (0.255 g, 0.784 mmol), oxalyl chloride (0.14 mL, 1.6 mmol), and ethanol (2 mL), following the procedure described in Step 3 of Example 1. Purification by flash chromatography using 30-50% chloroform in hexane as an eluant and drying at 55° C. yielded a brownish solid (0.243 g, 73%). ¹HNMR (300 MHz, DMSO-d₆): δ8.75 (s, 1H), 8.5 (s, 1H), 8.05 (d, 2H, J=9.2 Hz), 7.8 (d, 2H, J=7.7 Hz), 7.7 (q, 2H, J=8.5 Hz), 7.25-7.4 (m, 5H), 5.65 (s, 2H), 4.35 (q, 2H, J=7.2 Hz), 2.6 (s, 3H), and 1.35 ppm (t, 3H, J=7.3 Hz).

Step 3

### [5-(4-Acetylphenyl)-1-benzyl-1H-indol-3-yl](oxo)acetic acid

[5-(4-Acetylphenyl)-1-benzyl-1H-indol-3-yl](oxo)acetic acid was prepared from ethyl [5-(4-acetylphenyl)-1-benzyl-1H-indol-3-yl](oxo)acetate (0.225 g, 0.529 mmol), and potassium hydroxide (0.104 g, 1.85 mmol) in THF (7 mL) and water (7 mL) according to the procedure described in Step 4 of Example 2. After drying for 15 hours at 96° C., the title compound was obtained as a tan solid (0.166 g, 79%), mp: 213-214° C. (dec.). Mass spectrum (−APCI, [M−H]⁻), m/z 396; ¹HNMR (400 MHz, DMSO-d₆): δ8.75 (s, 1H), 8.5 (d, 1H, J=1.7 Hz), 8.05 (d, 2H, J=8.3 Hz), 7.8 (d, 2H, J=8.3 Hz), 7.7 (d, 1H, J=8.8 Hz), 7.65 (dd, 1H, J=8.7 Hz and 1.6 Hz), 7.25-7.4 (m, 5H), 5.65 (s, 2H), and 2.6 ppm (s, 3H). Elemental Analysis for C₂₅H₁₉NO₄.0.50H₂O: Calculated: C, 73.87; H, 4.96; N, 3.45. Found: C, 73.54; H, 4.64; N, 3.32.

### EXAMPLE 4

### {1-Benzyl-5-[4-(trifluoromethoxy)phenyl]-1H-indol-3-yl}(oxo)acetic acid

Step 1

### 1-Benzyl-5-[4-(trifluoromethoxy)phenyl]-1H-indole

1-Benzyl-5-[4-(trifluoromethoxy)phenyl]-1H-indole was prepared by coupling of 1-benzyl-5-bromo-1H-indole (5.2 g, 18 mmol) and 4-trifluoromethoxyphenylboronic acid (4.7 g, 23 mmol), using [1,1′-bis(diphenylphosphino)ferrocene]dichloropalladium (II) complex with dichloromethane (1:1) (0.88 g, 1.1 mmol), and potassium carbonate (3.8 g, 27 mmol) in dioxane (135 mL) and water (13.5 mL) the reaction according to the procedure outlined in Step 1 of Example 3. Purification by flash chromatography (Biotage apparatus) using hexane as an eluant yielded the title compound as a light yellow solid (2.8 g, 42%), mp: 62-63° C. ¹HNMR (300 MHz, DMSO-d₆): δ7.85 (s, 1H), 7.75 (d, 2H, J=7.7 Hz), 7.5-7.6 (m, 2H), 7.4 (d, 3H, J=7.7 Hz), 7.2-7.35 (m, 5H), 6.6 (d, 1H, J=3.9 Hz), and 5.45 ppm (s, 2H).

Step 2

### Ethyl 2-{1-benzyl-5-[4-(trifluoromethoxy)phenyl]-1H-indol-3-yl}-2-oxoacetate

Ethyl {1-benzyl-5-[4-(trifluoromethoxy)phenyl]-1H-indol-3-yl}(oxo)acetate was prepared from 1-benzyl-5-[4-(trifluoromethoxy)phenyl]-1H-indole (2.80 g, 7.62 mmol), oxalyl chloride (2.0 mL, 23 mmol), and ethanol (4.5 mL) according to the procedure described in Step 3 of Example 1. Purification by flash chromatography using 5-10% ethyl acetate in hexane as an eluant then drying at 60° C. furnished the title compound as a yellow gum (3.05 g, 86%). ¹HNMR (300 MHz, DMSO-d₆): δ8.75 (s, 1H), 8.45 (s, 1H), 7.8 (d, 2H, J=9.2 Hz), 7.75 (d, 1H, J=9.2 Hz), 7.6 (d, 1H, J=9.2 Hz), 7.45 (d, 2H, J=9.2 Hz), 7.3-7.4 (m, 5H), 5.85 (s, 2H), 4.35 (q, 2H, J=7.5 Hz), and 1.35 ppm (t, 3H, J=7.5 Hz).

Step 3

### {1-Benzyl-5-[4-(trifluoromethoxy)phenyl]-1H-indol-3-yl}(oxo)acetic acid

{1-Benzyl-5-[4-(trifluoromethoxy)phenyl]-1H-indol-3-yl}(oxo)acetic acid was prepared from ethyl 2-{1-benzyl-5-[4-(trifluoromethoxy)phenyl]-1H-indol-3-yl}-2-oxoacetate (0.463 g, 0.991 mmol), potassium hydroxide (0.224 g, 3.99 mmol) in THF (5 mL) and water (5 mL) according to the procedure described in Step 4 of Example 2. The title compound was obtained as a light yellow solid (0.314 g, 78%), mp 169-171° C. Mass spectrum (+APCI, [M+H]⁺), m/z 440; ¹HNMR (400 MHz, DMSO-d₆): δ13.8-14.2 (br s, 1H), 8.75 (s, 1H), 8.45 (d, 1H, J=1.5 Hz), 7.75-7.8 (m, 2H), 7.7 (d, 1H, J=8.5 Hz), 7.6 (dd, 1H, J=8.7 Hz), 7.45 (d, 2H, J=8.8 Hz), 7.25-7.35 (m, 5H), and 5.65 ppm (s, 2H). Elemental Analysis for C₂₄H₁₆F₃NO₄: Calculated: C, 65.61; H, 3.67; N, 3.19. Found: C, 65.59; H, 3.54; N, 3.17.

### EXAMPLE 5

### [1-(4-Methylbenzyl)-5-phenyl-1H-indol-3-yl](oxo)acetic acid

Step 1

### 5-Bromo-1-(4-methylbenzyl)-1H-indole

NaH (60%, 2.53 g, 63.1 mmol) was added portionwise to a stirring solution of 5-bromoindole (8.25 g, 42.1 mmol) in DMF (80 mL) at 0° C. under a nitrogen atmosphere over a period of 10 min. The mixture was then warmed up to room temperature. After the reaction mixture was stirred at room temperature for 1 hour, 4-methylbenzyl bromide (12.0 g, 63.1 mmol) was added and the mixture was stirred at room temperature overnight. The reaction was quenched with aqueous ammonium chloride and diluted with water. The aqueous phase was extracted with ethyl acetate. The organic extract was washed with water and brine, then dried over anhydrous magnesium sulfate. This mixture was concentrated to give a crude oil (14.1 g, 71%). Crystallization from petroleum ether afforded the title compound as a white solid, m.p: 56-57° C. Mass spectrum (APCI, [M+H]⁺), m/z 300. ¹HNMR (400 MHz, DMSO-d₆): δ7.73 (s, 1H), 7.53 (s, 1H), 7.40 (d, 1H, J=8.9 Hz), 7.18 (d, 1H, J=10.5 Hz), 7.09 (d, 2H, J=8.2 Hz), 7.07 (d, 2H, J=8.2 Hz), 6.45 (s, 1H), 5.35 (s, 2H), and 2.22 ppm (s, 3H). Elemental Analysis for C₁₆H₁₄BrN: Calculated: C, 64.02; H, 4.70; N, 4.67. Found: C, 63.66; H, 4.59; N, 4.71.

Step 2

### 1-(4-Methylbenzyl)-5-phenyl-1H-indole

A mixture of 5-bromo-1-(4-methylbenzyl)-1H-indole (1.0 g, 3.33 mmol), benzeneboronic acid (0.621 g, 5.0 mmol), potassium carbonate (0.691 g, 5.0 mmol), and [1′1′-bis(diphenylphosphino)-ferrocene]dichloropalladium(II) complex with methylenechloride (1:1) (0.816 g, 1.0 mmol) in dioxane-water (10:1, 16.5 mL) was stirred at 70° C. for 2 day. The reaction mixture was diluted with water and extracted with ethyl acetate. The organic extract was washed with water and brine, and then concentrated to an oil. The residue was purified by flash column chromatography using hexane/ethyl acetate (96:4) as an eluant to give the title compound as a semi-solid (0.48 g, 49%). Mass spectrum (+ESI, [M+H]⁺), m/z 298. ¹HNMR (300 MHz, DMSO-d⁶): δ7.80 (s, 1H), 7.63 (d, 2H, J=8.4 Hz), 7.48 (d, 2H, J=11.1 Hz), 7.38-7.45 (m, 3H), 7.11 (m, 4H), 6.52 (d, 2H, J=2.8 Hz), 5.38 (s, 2H), and 2.22 ppm (s, 3H). Elemental Analysis for C₂₂H₁₉N: Calculated: C, 88.85; H, 6.44; N, 4.71. Found: C, 88.65; H, 6.42; N, 4.61.

Step 3

### [1-(4-Methylbenzyl)-5-phenyl-1H-indol-3-yl](oxo)acetic acid

Oxalyl chloride (0.474 mL, 5.43 mmol) was added dropwise to a stirring solution of 1-(4-methylbenzyl)-5-phenyl-1H-indole (0.46 g, 1.55 mmol) in THF (15 mL) at room temperature over a period of 5 minutes under a nitrogen atmosphere. After the reaction mixture was stirred at room temperature for 4 hours, the reaction was quenched carefully with water. The aqueous mixture was extracted with ethyl acetate. The extract was washed with water, and brine, dried over anhydrous magnesium sulfate, and concentrated to give the title compound as a yellow solid (0.41 g, 72%), m.p: 195-196° C. Mass spectrum (ESI, [M+H]⁺), m/z 370. ¹HNMR (400 MHz, DMSO-d₆): δ 13.95 (br s, 1H), 8.68 (s, 1H), 8.41 (s, 1H), 7.63-7.66 (m, 3H), 7.56 (d, 1H, J=10.4 Hz), 7.47 (t, 2H, J=7.5 Hz), 7.35(t, 1H, J=7.3 Hz), 7.22(d, 2H, J=8.1 Hz), 7.15 (d, 2H, J=8.0 Hz), 5.56 (s, 2H), and 2.25 ppm (s, 3H). Elemental Analysis for C₂₄H₁₉NO₃.0.3 H₂O: Calculated: C, 76.91; H, 5.27; N, 3.74. Found: C, 76.85; H, 5.18; N, 3.61.

### EXAMPLE 6

### [1-(4-tert-Butylbenzyl)-5-(3-methylphenyl)-1H-indol-3-yl](oxo)acetic acid

Step 1

### 5-Bromo-1-[4-(tert-butyl)benzyl]-1H-indole

The title compound was prepared from 4-(tert-butyl)benzyl bromide (180 g, 768 mmol) and 5-bromoindole (152 g, 768 mmol) in substantially the same manner, as described in Step 1 of Example 5. The product (257 g, 97%) was obtained as a yellow solid, mp: 108-109° C. Mass spectrum (ESI, [M+H]+), m/z 342. 1HNMR (400 MHz, DMSO-d₆): δ 7.73 (s, 1H), 7.55 (s, 1H), 7.44 (d, 1H, J=8.71 Hz), 7.30 (d, 2H, J=7.96 Hz), 7.19 (d, 1H, J=8.71 Hz), 7.10 (d, 2H, J=7.63 Hz), 6.46 (s, 1H), 5.36 (s, 2H), and 1.21 ppm (s, 9H). Elemental Analysis for C₁₉H₂₀BrN: Calculated: C, 66.67; H, 5.89; N, 4.09. Found: C, 66.78; H, 5.86; N, 4.02.

Step 2

### 1-[4-(tert-Butyl)benzyl]-5-(3-methylphenyl)-1H-indole

The mixture of 5-bromo-1-(4-tert-butylbenzyl)-1H-indole (67.5 g, 197.2 mmol), 3-methylbenzeneboronic acid (27.6 g, 197.2 mmol), potassium carbonate (27.2 g, 493 mmol), palladium(II) acetate (0.338 g) and tetrabutylammonium bromide (63.5 g, 197.2 mmol) in 10% dioxane in water (degassed, 1.72 L) was stirred at 70° C. The reaction was monitored by TLC. 3-Methylbenzeneboronic acid (45.2 g, 394.4 mmol) was added in four portions every 10 hours, after which time 5-bromo-1-(4-tert-butylbenzyl)-1H-indole was no longer detected by TLC. The reaction was cooled to room temperature and the solvent was decanted. The dark gum-like oil was washed with water and extracted with petroleum ether (4×2 L). The combined petroleum ether extracts were washed with water and filtered. This mixture was concentrated to a volume of about 1.5 L and allowed to crystallize. The solid was isolated by filtration and dried under vacuum at 60° C. for 10 hours. to afford the title compound as a white solid (50.8 g, 73%), mp: 94-95° C. Mass spectrum (ESI, [M+H]⁺), m/z 354. ¹HNMR (400 MHz, DMSO-d₆): δ 7.79 (s, 1H), 7.53-7.51 (m, 2H), 7.45 (s, 1H), 7.41 (d, 1H, J=7.79 Hz), 7.37 (d, 2H, J=8.55 Hz), 7.32-7.28 (m, 3H), 7.14 (d, 2H, J=8.40 Hz), 7.09 (d, 1H, J=8.40 Hz), 6.51 (d, 1H, J=2.75 Hz), 5.38 (s, 2H), 2.36 (s, 3H), and 1.21 ppm (s, 9H). Elemental Analysis for C₂₆H₂₇N: Calculated: C, 88.34; H, 7.70; N, 3.96. Found: C, 88.24; H, 7.64; N, 3.92.

Step 3

### [1-(4-tert-Butylbenzyl)-5-(3-methylphenyl)-1H-indol-3-yl](oxo)acetic acid

The title compound was prepared from 1-[4-(tert-butyl)benzyl]-5-(3-methylphenyl)-1H-indole (44.6 g, 126.2 mmol) and oxalyl chloride (22.0 mL, 252.4 mmol) in substantially the same manner, as described in Step 3 of Example 5. The product was obtained as a yellow solid (51.4 g, 96%), mp: 128-129° C. Mass spectrum (ESI, [M−H]⁻), m/z 424. 1HNMR (400 MHz, DMSO-d₆): δ 13.50 (br s, 1H), 8.70 (s, 1H), 8.40 (s, 1H), 7.69 (d, 1H, J=8.71 Hz), 7.56 (d, 1H, J=8.55 Hz), 7.45-7.42 (m, 2H), 7.37-7.33 (m, 3H), 7.24 (d, 2H, J=8.40 Hz), 7.16 (d, 1H, J=7.49 Hz), 5.57 (s, 2H), 2.38 (s, 3H), and 1.22 ppm (s, 9H). Elemental Analysis for C₂₈H₂₇NO₃: Calculated: C, 79.03; H, 6.40; N, 3.29. Found: C, 78.77; H, 6.29; N, 3.25.

### EXAMPLE 7

### PAI-1 Inhibitors for the Treatment of Muscular Disorders

The mdx mouse is a genetic model of Duchenne and Becker muscular dystrophies, providing a model system of muscle degeneration with aberrant regeneration. In this model, a nonsense mutation in the dystrophin gene results in the absence of dystrophin from the sarcolemma, and subsequent muscle fiber necrosis. Conversely, mice deficient in plasminogen activator inhibitor-1 have improved skeletal muscle regeneration. It was hypothesized that a small molecule PAI-1 inhibitor, such as, for example {1-Benzyl-5-[4-(trifluoromethoxy)phenyl]-1H-indol-3-yl}(oxo)acetic acid could potentially produce beneficial effects on the skeletal muscle of mdx mice.

Female mdx mice were purchased from a commercial vendor, and divided into 2 groups of equal body weight. {1-Benzyl-5-[4-(trifluoromethoxy)phenyl]-1H-indol-3-yl}(oxo)acetic acid was formulated into rodent chow at a concentration of 1 mg of drug per 1 gram of food (1 mg/g), and this mixture was formed into food pellets. A standard, pelleted rodent chow was used as the control diet. One group of mice received the drug-containing diet, while the other group received the control diet ad libitum. Each diet was coded, and the animal technicians remained blinded to the composition of the diets throughout the study.

Eight mice received either the control or drug-containing diet for 9 months. At the end of the study, the mice were killed, and muscle and body weights were measured. There was no change in body weight between groups (28.1±0.45 g for drug-treated vs. 28.3±0.69 g for Control). Treatment with the PAI-1 inhibitor resulted in a greater gastrocnemius muscle mass, however, as drug-treated mice exhibited a muscle weight of 141.0±4.1 mg compared to 128.9±5.9 mg for control mice (p=0.11, 8.6% difference in group means). Because muscular dystrophy is a wasting disease, changes in muscle weight are routinely expressed per unit of body weight in preclinical studies. Therefore, the individual weight of each gastrocnemius muscle (mg) was expressed relative to total body weight (g). When expressed in these units, drug treatment also resulted in an increased muscle weight compared to muscle weight of mice feeding on normal chow. In drug-treated mice, muscle weight per unit of body weight was 5.02±0.13 mg/g, while in mice on the control diet, this value was 4.56 mg/g±0.20 (p=0.07). Taken together, these data indicate that treatment with the synthetic PAI-1 inhibitor {1-Benzyl-5-[4-(trifluoromethoxy)phenyl]-1H-indol-3-yl}(oxo)acetic acid resulted in an increased relative ratio of skeletal muscle weight to body weight in a murine model of muscular dystrophy.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed:

1. A method of treating muscle damage, muscle wasting, muscle degeneration, muscle atrophy or reduced rate of muscle repair, wherein said method comprises administering an effective amount of a compound of formula (I), or a pharmaceutically acceptable salt, solvate or ester thereof, to a mammal in need thereof: 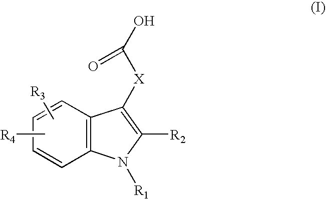
wherein:
X is a chemical bond, —CH₂— or —C(O)—;
R₁ is C₁-C₈ alkyl, (—CH₂)ₙ—C₃-C₆ cycloalkyl, wherein n is an integer of from 0 to 3, pyridinyl, —CH₂-pyridinyl, phenyl or benzyl, the rings of the cycloalkyl, pyridinyl, phenyl and benzyl groups being optionally substituted by, from 1 to 3 groups selected from, halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is H, C₁-C₆ alkyl, C₃-C₆ cycloalkyl, —CH₂—C₃-C₆ cycloalkyl, C₁-C₃ perfluoroalkyl, —CH₂OH or CH₂OAc;
R₃ is H, halogen, C₁-C₆ alkyl, C₁-C₃ perfluoroalkyl, C₁-C₆ alkoxy, C₃-C₆ cycloalkyl, —CH₂—C₃-C₆ cycloalkyl, C₃-C₆ cycloalkenyl, —CH₂—C₃-C₆ cycloalkenyl, —NH₂, or —NO₂; and
R₄ is C₃-C₆ cycloalkyl, —CH₂—C₃-C₆ cycloalkyl, C₃-C₆ cycloalkenyl, —CH₂—C₃-C₆ cycloalkenyl, phenyl, benzyl, pyridinyl, or —CH₂-pyridinyl, with the rings of these groups being optionally substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, —NO₂ or (CO)C₁-C₆ alkyl.

2. The method of claim 1 wherein the compound of formula (I) is a compound of formula (II) or a pharmaceutically acceptable salt, solvate or ester thereof:

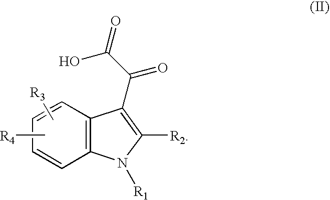

3. The method of claim 1 wherein the compound of formula (I) is a compound of formula (III) or (IV) or a pharmaceutically acceptable salt, solvate or ester thereof

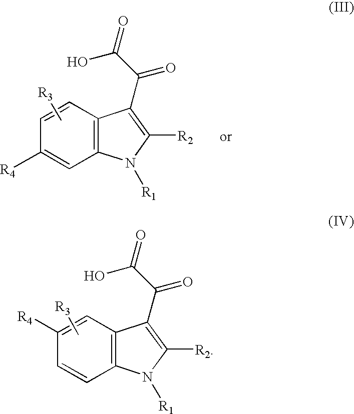

4. The method of claim 1, wherein the compound of formula (I) is a compound of formula (V) or formula (VI), or a pharmaceutically acceptable salt, solvate or ester thereof: 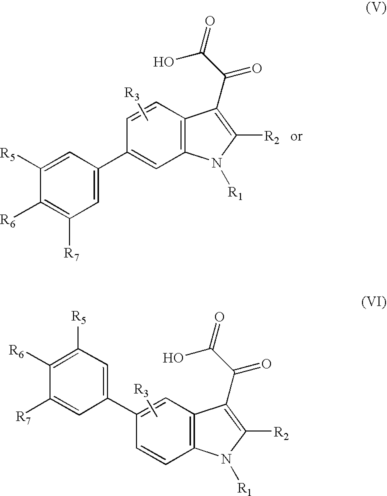
wherein:
R₁ is C₁-C₈ alkyl, C₃-C₆ cycloalkyl, —CH₂—C₃-C₆ cycloalkyl, or benzyl, the rings of the cycloalkyl and benzyl groups being optionally substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is H, C₁-C₆ alkyl, C₃-C₆ cycloalkyl, —CH₂—C₃-C₆ cycloalkyl, or C₁-C₃ perfluoroalkyl;
R₃ is H, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂; and
R₅, R₆ and R₇ are independently selected from H, halogen, C₁-C₃ alkyl, C₁-C₃ perfluoroalkyl, —O—C₁-C₃ perfluoroalkyl, C₁-C₃ alkoxy, —OH, —NH₂, or —NO₂.

5. The method of claim 1, wherein the compound of formula (I) is a compound of formula (VI), or a pharmaceutically acceptable salt, solvate or ester thereof: 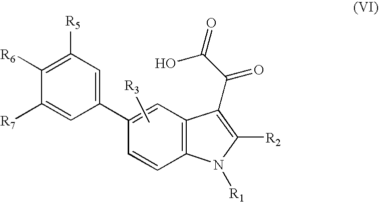
wherein:
R₁ is benzyl, the benzyl group being optionally substituted by from 1 to 3 groups selected from halogen, C₁-C₄ alkyl, C₁-C₃ perfluoroalkyl, —O—C₁-C₃ perfluoroalkyl, or C₁-C₃ alkoxy;
R₂ is H;
R₃ is H; and
R₅, R₆ and R₇ are independently H, halogen, C₁-C₃ alkyl, C₁-C₃ perfluoroalkyl, —O—C₁-C₃ perfluoroalkyl or C₁-C₃ alkoxy.

6. The method of claim 1, wherein the compound of formula (I) is a compound of formula (VI), or a pharmaceutically acceptable salt, solvate or ester thereof: 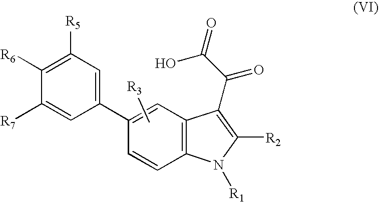
wherein:
R₁ is C₁-C₈ alkyl, (—CH₂)ₙ—C₃-C₆ cycloalkyl, wherein n is an integer of from 0 to 3, or benzyl, the rings of the cycloalkyl, pyridinyl, phenyl and benzyl groups being optionally substituted by, from 1 to 3 groups selected from, halogen, C₁-C₄ alkyl, C₁-C₃ perfluoroalkyl, —O—C₁-C₃ perfluoroalkyl, or C₁-C₃ alkoxy;
R₂ is H, —CH₂OH or CH₂OAc;
R₃ is H;
R₅, R₆ and R₇ are independently H, halogen, C₁-C₃ alkyl, C₁-C₃ perfluoroalkyl, —O—C1-C₃ perfluoroalkyl C₁-C₃ alkoxy or (CO)C₁-C₆ alkyl.

7. The method of claim 1 wherein at least one of R₅, R₆ and R₇ is not H.

8. The method of claim 1, wherein the compound of formula (I) is
a) {1-Methyl-6-[4-(trifluoromethoxy)phenyl]-1H-indol-3-yl}(oxo)acetic acid;
b) {1-Methyl-6-[4-(trifluoromethyl)phenyl]-1H-indol-3-yl}(oxo)acetic acid;
c) {1-Ethyl-6-[4-(trifluoromethoxy)phenyl]-1H-indol-3-yl}(oxo)acetic acid;
d) {1-Ethyl-6-[4-(trifluoromethyl)phenyl]-1H-indol-3-yl}(oxo)acetic acid;
e) {1-Benzyl-6-[4-(trifluoromethoxy)phenyl]-1H-indol-3-yl}(oxo)acetic acid;
f) {1-Benzyl-6-[4-(trifluoromethyl)phenyl]-1H-indol-3-yl}(oxo)acetic acid;
g) {1-[4-(tert-Butyl)benzyl]-6-[4-(trifluoromethyl)phenyl]-1H-indol-3-yl}(oxo)acetic acid;
h) {1-[4-(tert-Butyl)benzyl]-6-[4-(trifluoromethoxy)phenyl]-1H-indol-3-yl}(oxo)acetic acid;
i) {1-Benzyl-5-[4-(trifluoromethyl)phenyl]-1H-indol-3-yl}(oxo)acetic acid;
j) {6-[4-(tert-Butyl)phenyl]-1-methyl-1H-indol-3-yl}(oxo)acetic acid;
k) [5-(4-Acetylphenyl)-1-benzyl-1H-indol-3-yl](oxo)acetic acid;
l) {1-Benzyl-5-[4-(trifluoromethoxy)phenyl]-1H-indol-3-yl}(oxo)acetic acid;
m) {1-Benzyl-4-[4-(trifluoromethyl)phenyl]-1H-indol-3-yl}(oxo)acetic acid;
n) {1-Benzyl-5-[4-(tert-butyl)phenyl]-1H-indol-3-yl}(oxo)acetic acid;
o) [1-Benzyl-5-(3-chloro-4-fluorophenyl)-1H-indol-3-yl](oxo)acetic acid;
p) {1-Benzyl-5-[3,5-bis(trifluoromethyl)phenyl]-1H-indol-3-yl}(oxo)acetic acid;
q) {1-Benzyl-7-[4-(trifluoromethoxy)phenyl]-1H-indol-3-yl}(oxo)acetic acid;
r) [1-Benzyl-7-(3-chloro-4-fluorophenyl)-1H-indol-3-yl](oxo)acetic acid;
s) {1-(4-tert-Butylbenzyl)-5-[4-(trifluoromethoxy)phenyl]-1H-indol-3-yl}(oxo)acetic acid;
t) {1-Benzyl-4-[4-(trifluoromethoxy)phenyl]-1H-indol-3-yl}(oxo)acetic acid;
u) [1-Benzyl-6-(3-chlorophenyl)-1H-indol-3-yl](oxo)acetic acid;
v) {1-Benzyl-5-[3-(trifluoromethoxy)phenyl]-1H-indol-3-yl}(oxo)acetic acid;
w) {1-(4-Methylbenzyl)-5-[4-(trifluoromethoxy)phenyl]-1H-indol-3-yl}(oxo)acetic acid;
x) {1-(4-Fluorobenzyl)-5-[4-(trifluoromethoxy)phenyl]-1H-indol-3-yl}(oxo)acetic acid;
y) [1-Butyl-5-(4-chlorophenyl)-1H-indol-3-yl](oxo)acetic acid;
z) [1-Butyl-5-(3-chlorophenyl)-1H-indol-3-yl](oxo)acetic acid;
aa) [1-Butyl-5-(3-methoxyphenyl)-1H-indol-3-yl](oxo)acetic acid;
bb) [1-Butyl-5-(4-methoxyphenyl)-1H-indol-3-yl](oxo)acetic acid;
cc) {1-Butyl-5-[4-(trifluoromethyl)phenyl]-1H-indol-3-yl}(oxo)acetic acid;
dd) [1-(4-tert-Butylbenzyl)-5-(3-methylphenyl)-1H-indol-3-yl](oxo)acetic acid;
ee) [1-(4-tert-Butylbenzyl)-5-(3-methoxyphenyl)-1H-indol-3-yl](oxo)acetic acid;
ff) [1-(4-tert-Butylbenzyl)-5-(4-tert-butylphenyl)-1H-indol-3-yl](oxo)acetic acid;
gg) [1-(4-tert-Butylbenzyl)-5-(3-chlorophenyl)-1H-indol-3-yl](oxo)acetic acid;
hh) [1-(4-tert-Butylbenzyl)-5-(4-chlorophenyl)-1H-indol-3-yl](oxo)acetic acid;
ii) [1-(4-tert-Butylbenzyl)-5-(2-methylphenyl)-1H-indol-3-yl](oxo)acetic acid;
jj) {1-(2-Ethylbutyl)-5-[4-(trifluoromethoxy)phenyl]-1H-indol-3-yl}(oxo)acetic acid;
kk) {2-[(Acetyloxy)methyl]-1-(4-methylbenzyl)-5-[4-(trifluoromethoxy)phenyl]-1H-indol-3-yl}(oxo)acetic acid;
ll) {2-(Hydroxymethyl)-1-(4-methylbenzyl)-5-[4-(trifluoromethoxy)phenyl]-1H-indol-3-yl}(oxo)acetic acid;
mm) {2-[(Acetyloxy)methyl]-1-benzyl-5-[4-(trifluoromethoxy)phenyl]-1H-indol-3-yl}(oxo)acetic acid;
nn) {1-Benzyl-2-(hydroxymethyl)-5-[4-(trifluoromethoxy)phenyl]-1H-indol-3-yl}(oxo)acetic acid;
oo) [5-(3-Chlorophenyl)-1-cyclopentyl-1H-indol-3-yl]-oxo-acetic acid;
pp) [5-(3-chlorophenyl)-1-(cyclobutylmethyl)-1H-indol-3-yl](oxo)acetic acid;
qq) [5-(3-chlorophenyl)-1-(3-methylcyclopropyl)-1H-indol-3-yl](oxo)acetic acid;
rr) [5-(3-chlorophenyl)-1-(cyclohexylmethyl)-1H-indol-3-yl](oxo)acetic acid;
ss) [5-(4-trifluoromethylphenyl)-1-(cyclopentyl)-1H-indol-3-yl](oxo)acetic acid;
tt) [5-(4-trifluoromethylphenyl)-1-(cyclobutylmethyl)-1H-indol-3-yl](oxo)acetic acid;
uu) [5-(4-trifluoromethylphenyl)-1-(3-methylcyclopentyl)-1H-indol-3-yl](oxo)acetic acid;
vv) [5-(4-trifluoromethylphenyl)-1-(cyclohexylmethyl)-1H-indol-3-yl](oxo)acetic acid;
ww) [5-(4-trifluoromethylphenyl)-1-(cyclopentylpropyl)-1H-indol-3-yl](oxo)acetic acid;
xx) [5-(3-trifluoromethylphenyl)-1-(cyclopentyl)-1H-indol-3-yl](oxo)acetic acid;
yy) [5-(3-trifluoromethylphenyl)-1-(cyclobutylmethyl)-1H-indol-3-yl](oxo)acetic acid;
zz) [5-(3-trifluoromethylphenyl)-1-(3-methylcyclopentyl)-1H-indol-3-yl](oxo)acetic acid;
aaa) [5-(3-trifluoromethylphenyl)-1-(cyclohexylmethyl)-1H-indol-3-yl](oxo)acetic acid;
bbb) [5-(3-trifluoromethylphenyl)-1-(cyclopentylpropyl)-1H-indol-3-yl](oxo)acetic acid; or
ccc) [5-(4-methoxyphenyl)-1-(cyclohexylmethyl)-1H-indol-3-yl](oxo)acetic acid; or a pharmaceutically acceptable salt, solvate or ester thereof.

9. A method of treating muscle damage, muscle wasting, muscle degeneration, muscle atrophy or reduced rate of muscle repair, wherein said method comprises administering an effective amount of a compound of formula (VII), or a pharmaceutically acceptable salt, solvate or ester thereof, to a mammal in need thereof: 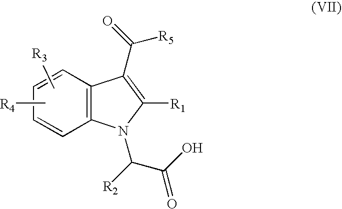
wherein:
R₁ is hydrogen, C₂-C₆ alkyl, C₃-C₆ cycloalkyl, —CH₂—C₃-C₆ cycloalkyl, or C₁-C₃ perfluoroalkyl, wherein the alkyl and cycloalkyl groups are optionally substituted with halogen, —CN, C₁-C₆ alkoxy, —OH, —NH₂, or —NO₂;
R₂ is hydrogen, C₁-C₈ alkyl, C₃-C₆ cycloalkyl, —CH₂—C₃-C₆ cycloalkyl, thienyl, CH₂-thienyl, furanyl, CH₂-furanyl, oxazoyl, CH₂-oxazoyl, phenyl, benzyl, CH₂-naphthyl, wherein the alkyl group and the rings of the cycloalkyl, thienyl, furanyl, oxazoyl, phenyl, benzyl, and napthyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, —CH₂CO₂H, —C(O)CH₃, —CO₂R₆, —C(O)NH₂, —S(O)₂CH₃, —OH, —NH₂, or —NO₂;
R₃ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂;
R₄ is C₃-C₈ alkyl, C₃-C₆ alkenyl, C₃-C₆ alkynyl, C₃-C₆ cycloalkyl, —CH₂—C₃-C₆ cycloalkyl, thienyl, furanyl, oxazoyl, phenyl, benzo[b]furan-2-yl, benzo[b]thien-2-yl, benzo[1,3]dioxol-5-yl, naphthyl, wherein the alkyl groups and the rings of the cycloalkyl, thienyl, furanyl, oxazoyl, phenyl, benzofuranyl, benzothienyl, and napthyl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —COOH, CH₂CO₂H, —C(O)CH₃, —C(O)OR₆, —C(O)NH₂, —S(O)₂CH₃, —OH, —NH₂, or —NO₂;
R₅ is C₁-C₈ alkyl, C₃-C₆ cycloalkyl, —CH₂—C₃-C₆ cycloalkyl, pyridinyl, —CH₂-pyridinyl, thienyl, CH₂-thienyl, furanyl, CH₂-furanyl, oxazoyl, CH₂-oxazoyl, phenyl, benzyl, benzo[b]furan-2-yl, benzo[b]thien-2-yl, benzo[1,3]dioxol-5-yl, naphthyl, CH₂-naphthyl, 9H-fluoren-1-yl, 9H-fluoren-4-yl, 9H-fluoren-9-yl, 9-fluorenone-1-yl, 9-fluorenone-2-yl, 9-fluorenone-4-yl, CH₂-9H-fluoren-9-yl, wherein the alkyl group and the rings of the cycloalkyl, pyridinyl, thienyl, furanyl, oxazoyl, phenyl, benzyl, benzofuranyl, benzothienyl, napthyl, fluorenyl, and fluorenone groups are optionally substituted by from 1 to 3 groups selected from halogen, C₁-C₃ alkyl, C₃-C₆ cycloalkyl, C₁-C₃ perfluoroalkyl, —O—C₁-C₃ perfluoroalkyl, —S—C₁-C₃ perfluoroalkyl, C₁-C₃ alkoxy, phenoxy, —OCHF₂, —CN, —COOH, —CH₂CO₂H, —C(O)CH₃, —CO₂R₆, —C(O)NH₂, —S(O)₂CH₃, —OH, —NH₂, or —NO₂, wherein the phenoxy group are optionally substituted by from 1 to 3 groups selected from halogen, C₁-C₃ alkyl, or C₁-C₃ perfluoroalkyl; and
R₆ is C₁-C₆ alkyl, C₃-C₆ cycloalkyl, —CH₂—C₃-C₆ cycloalkyl, or benzyl.

10. The method of claim 9 wherein the compound of Formula (VIII) is
(a) [3-(4-chlorobenzoyl)-5-(4-chlorophenyl)-1H-indol-1-yl]acetic acid;
(b) [3-(Benzo[b]thiophene-2-carbonyl)-5-(4-methylphenyl)-1H-indol-1-yl]-acetic acid; or
(c) [3-(4-chlorobenzoyl)-5-(4-methylphenyl)-1H-indol-1-yl]-acetic acid; or a pharmaceutically acceptable salt, solvate, or ester form thereof.

11. A method of treating muscle damage, muscle wasting, muscle degeneration, muscle atrophy or reduced rate of muscle repair, wherein said method comprises administering an effective amount of a compound of formula (XI), or a pharmaceutically acceptable salt, solvate or ester thereof, to a mammal in need thereof: 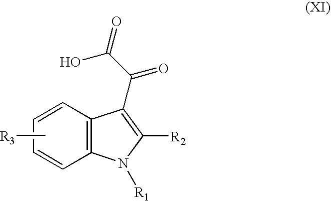
wherein:
R₁ is the moiety: 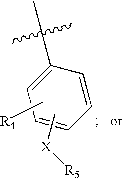
R₁ is C₁-C₈ alkyl, benzo[1,3]dioxo-5yl-methyl, cycloalkylalkyl where the alkyl chain is C₁-C₃, heteroarylalkyl where the alkyl chain is C₁-C₃, arylalkyl where the alkyl chain is C₁-C₃, preferably selected from benzyl, CH₂-1-naphthyl, CH₂-2-naphyl, CH₂CH₂-phenyl, or CH₂CH₂-napthyl, wherein the alkyl, cycloalkyl, heteroaryl, and aryl groups are optionally substituted by from 1 to 3 groups selected from halogen, C₁-C₃ alkyl, C₁-C₃ haloalkyl, C₁-C₃ perfluoroalkyl, C₁-C₃ alkoxy, C₁-C₃ perfluoroalkoxy, C₁-C₃ alkylthio, C₁-C₃ perfluoroalkylthio, —OCHF₂, —CN, —C(O)CH₃, —CO₂R₇, —C(O)NH₂, —S(O)₂CH₃, —OH, —NH₂, or —NO₂;
R₄ is hydrogen, halogen, C₁-C₃ alkyl, C₁-C₃ haloalkyl, C₁-C₃ perfluoroalkyl, C₁-C₃ alkoxy, C₁-C₃ perfluoroalkoxy, C₁-C₃ alkylthio, C₁-C₃ perfluoroalkylthio, —OCHF₂, —CN, —COOH, —CH₂CO₂H, —C(O)CH₃, —CO₂R₇, —C(O)NH₂, —S(O)₂CH₃, —OH, —NH₂, or —NO₂;
X is O, S, or NH;
R₅ is C₁-C₈ alkyl, C₁-C₃ perfluoroalkyl, C₃-C₆ cycloalkyl, —CH₂—C₃-C₆ cycloalkyl, heteroaryl, —CH₂-heteroaryl, phenyl, or arylalkyl where the alkyl chain is C₁-C₈, wherein the rings of the cycloalkyl, heteroaryl, phenyl, and aryl groups are optionally substituted by from 1 to 5 groups selected from halogen, C₁-C₆ alkyl, C₁-C₃ haloalkyl, C₁-C₃ perfluoroalkyl, C₁-C₃ alkoxy, C₁-C₃ perfluoroalkoxy, C₁-C₃ alkylthio, C₁-C₃ perfluoroalkylthio, heteroaryl, —OCHF₂, —CN, —COOH, —CH₂CO₂H, —C(O)CH₃, —CO₂R₇, —C(O)NH₂, —S(O)₂CH₃, —OH, —NH₂, or —NO₂;
R₂ is hydrogen, C₁-C₆ alkyl, —CH₂—C₃-C₆ cycloalkyl, or C₁-C₃ perfluoroalkyl, wherein the alkyl and cycloalkyl groups are optionally substituted by halogen, —CN, C₁-C₆ alkoxy, —COOH, —CH₂CO₂H, —C(O)CH₃, —CO₂R₇, —C(O)NH₂, —S(O)₂CH₃, —OH, —NH₂, or —NO₂;
R₃ is hydrogen, halogen, C₁-C₈ alkyl, C₁-C₈ alkenyl, C₁-C₈ alkynyl, C₃-C₆ cycloalkyl, —CH₂—C₃-C₆ cycloalkyl, heteroaryl, or phenyl, wherein the alkyl, alkenyl, alkynyl, cycloalkyl, heteroaryl, and phenyl groups are optionally substituted by from 1 to 3 groups selected from halogen, C₁-C₃ alkyl, C₁-C₃ haloalkyl, C₁-C₃ perfluoroalkyl, C₁-C₃ alkoxy, C₁-C₃ perfluoroalkoxy, C₁-C₃ alkylthio, C₁-C₃ perfluoroalkylthio, —OCHF₂, —CN, —COOH, —CH₂CO₂H, —C(O)CH₃, —CO₂R₇, —C(O)NH₂, —S(O)₂CH₃, —OH, —NH₂, or —NO₂;
or R₃ is the moiety X—R₆;
R₆ is C₁-C₈ alkyl, C₁-C₈ alkenyl, C₁-C₈ alkynyl, C₃-C₆ cycloalkyl, —CH₂—C₃-C₆ cycloalkyl, heteroaryl, phenyl, aryl-alkyl where the alkyl chain is C₁-C₈, CH₂CH₂-phenyl, or CH₂CH₂-napthyl, wherein the alkyl, alkenyl, alkynyl, cycloalkyl, heteroaryl, and aryl groups are optionally substituted by from 1 to 3 groups selected from halogen, C₁-C₃ alkyl, C₁-C₃ perfluoroalkyl, —O—C₁-C₃ perfluoroalkyl, —S—C₁-C₃ perfluoroalkyl, C₁-C₃ alkoxy, —OCHF₂, —CN, —C(O)CH₃, —CO₂R₇, —S(O)₂CH₃, —OH, —NH₂, or —NO₂; and
R₇ is C₁-C₆ alkyl, C₃-C₆ cycloalkyl, —CH₂—C₃-C₆ cycloalkyl, or C₁-C₈ aryl-alkyl.

12. The method of claim 11 wherein the compound of formula (XI) is
(a) (1-{4-[(4-cyanobenzyl)oxy]phenyl}-1H-indol-3-yl)(oxo)acetic acid;
(b) {1-[4-(3-methoxy-benzyloxy)-phenyl]1H-indol-3-yl}-oxo-acetic acid;
(c) {1-[4-(3-chloro-benzyloxy)-phenyl]1H-indol-3-yl}-oxo-acetic acid;
(d) {1-[4-(4-cyanobenzyloxy)-phenyl]-5-fluoro-1H-indol-3-yl}-oxo-acetic acid;
(e) {1-[4-(3,5-dimethoxy-benzyloxy)-phenyl]-5-fluoro-1H-indol-3-yl}-oxo-acetic acid;
(f) {1-[4-(3-chloro-benzyloxy)-phenyl]-5-methyl-1H-indol-3-yl}-oxo-acetic acid;
(g) {1-[4-(2,4-dichlorobenzyloxy)-phenyl]-5-methyl-1H-indol-3-yl}-oxo-acetic acid;
(h) {5-Chloro-1-[3-(4-cyano-benzyloxy)-phenyl]1H-indol-3-yl}-oxo-acetic acid;
(i) {5-Chloro-1-[3-(3,5-dimethoxy benzyloxy)-phenyl]1H-indol-3-yl}-oxo-acetic acid;
(j) {1-[4-(2,3,5,6-tetrafluoro-4-trifluoromethyl-benzyloxy)-phenyl]1H-indol-3-yl}-oxo-acetic acid;
(j) {1-[4-(2,6-dichloro-pyridin-4-ylmethoxy)-phenyl]1H-indol-3-yl}-oxo-acetic acid;
(k) [1-(4-{[5-(ethoxycarbonyl)-2-furyl]methoxy}phenyl)-5-fluoro-1H-indol-3-yl](oxo)acetic acid;
(l) {1-[4-(2,6-dichloropyridin-4-ylmethoxy)-phenyl]-5-methyl-1H-indol-3-yl}oxo-acetic acid;
(m) {5-Chloro-1-[3-(2,3,5,6-tetrafluoro-4-trifluoromethyl-benzyloxy)-phenyl]1H-indol-3-yl}-oxo-acetic acid;
(n) [5-chloro-1-(3-{[5-(ethoxycarbonyl)-2-furyl]methoxy}phenyl)-1H-indol-3-yl](oxo)acetic acid;
(o) 5-Chloro-1-[3-(2,6-dichloro-pyridin-4-ylmethoxy)-phenyl]1H-indol-3-yl}-oxo-acetic acid;
(p) [1,5-bis-(4-trifluoromethoxy-phenyl)-1H-indol-3-yl]-oxo-acetic acid;
(q) [1,5-bis-(4-trifluoromethoxy-phenyl)-1H-indol-3-yl]-oxo-acetic acid;
(r) {1-(4-fluorobenzyl)-5-[2-(4-fluorophenyl)ethoxy]-1H-indol-3-yl}(oxo)acetic acid;
(s) [1-benzyl-5-(2-chloro-4-trifluoromethyl-phenoxy)-1H-indol-3-yl](oxo)acetic acid;
(t) (1-benzyl-5-benzyloxy-1H-indol-3-yl)-oxo-acetic acid;
(u) (5-allyloxy-1-cyclobutylmethyl-1H-indol-3-yl)-oxo-acetic acid;
(v) (5-allyloxy-1-phenethyl-1H-indol-3-yl)-oxo-acetic acid;
(w) (5-allyloxy-1-benzo[1,3]dioxol-5-ylmethyl-1H-indol-3-yl)-oxo-acetic acid;
(x) (5-allyloxy-1-[2-(4-methoxyphenyl)-ethyl]-1H-indol-3-yl)-oxo-acetic acid;
(y) (5-allyloxy-1-[2-naphthalene-1-yl-ethyl]-1H-indol-3-yl)-oxo-acetic acid;
(z) (5-allyloxy-1-[2-(3-trifluoromethylphenyl)-ethyl]-1H-indol-3-yl)-oxo-acetic acid;
(aa) (5-allyloxy-1-[2-(4-bromophenyl)-ethyl]-1H-indol-3-yl)-oxo-acetic acid;
(bb) {1-[4-(4-tert-butyl-benzyloxy)-phenyl]-5-methyl-1H-indol-3-yl}-oxo-acetic acid;
(cc) {1-[4-(4-[1,2,3]thiadiazol-4-yl-benzyloxy)-phenyl]-1H-indol-3-yl}-oxo-acetic acid; or
(dd) {5-Chloro-1-[3-(4-[1,2,3]thiadiazol-4-yl-benzyloxy)-phenyl]1H-indol-3-yl}-oxo-acetic acid; or a pharmaceutically acceptable salt, solvate, or ester form thereof.

13. A method of treating muscle damage, muscle wasting, muscle degeneration, muscle atrophy or reduced rate of muscle repair, wherein said method comprises administering an effective amount of a compound of formula (XIV) or (XV), or a pharmaceutically acceptable salt, solvate or ester thereof, to a mammal in need thereof: 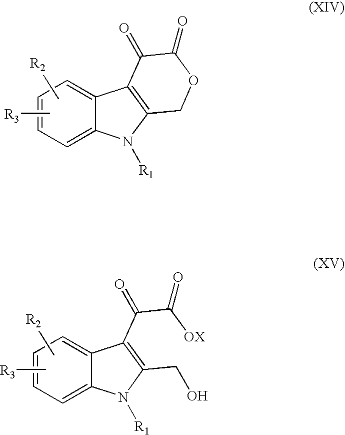
wherein:
X is hydrogen, an alkali metal or a basic amine moiety;
R₁ is hydrogen, C₁-C₈ alkyl, C₃-C₆ cycloalkyl, —CH₂—C₃-C₆ cycloalkyl, pyridinyl, —CH₂-pyridinyl, phenyl or benzyl, wherein the rings of the cycloalkyl, pyridinyl, phenyl and benzyl groups are optionally substituted by from 1 to 3 groups selected from halogen, C₁-C₆ alkyl, C₁-C₆ perfluoroalkyl, —O—C₁-C₆ perfluoroalkyl, C₁-C₆ alkoxy, —OH, —NH₂, or —NO₂;
R₂ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, hydroxy, —NH₂, or —NO₂; and
R₃ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, hydroxy, —NH₂, —NO₂, phenyl, benzyl, benzyloxy, pyridinyl, or —CH₂-pyridinyl, wherein the rings of these groups are optionally substituted by from 1 to 3 groups selected from phenyl, halogen, C₁-C₆ alkyl, C₁-C₆ perfluoroalkyl, —O—C₁-C₆ perfluoroalkyl, C₁-C₆ alkoxy, —OH, —NH₂, or —NO₂.

14. The method of claim 13 wherein the compound of formula (XIV) or (XV) is
(a) 9-(4-Methylbenzyl)-6-[4-(trifluoromethoxy)phenyl]-1,9-dihydropyrano[3,4-b]indole-3,4-dione;
(b) 9-Benzyl-6-[4-(trifluoromethoxy)phenyl]-1,9-dihydropyrano[3,4-b]indole-3,4-dione;
(c) 9-(4-Methylbenzyl)-6-(3-Methylphenyl)-1,9-dihydropyrano[3,4-b]indole-3,3-dione;
(d) 9-(4-tert-butylbenzyl)-6-(3-Methylphenyl)-1,9-dihydropyrano[3,4-b]indole-3,4-dione;
(e) 6-(Benzyloxy)-9-(4-methylbenzyl)-1,9-dihydropyrano[3,4-b]indole-3,4-dione;
(f) 6-(Benzyloxy)-1,9-dihydropyrano[3,4-b]indole-3,4-dione;
(g) 6-(Benzyloxy)-9-(4-tertbutylbenzyl)-1,9-dihydropyrano[3,4-b]indole-3,4-dione;
(h) 9-(4-tertbutybenzyl)-6-hydroxy-1,9-dihydropyrano[3,4-b]indole-3,4-dione;
(i) 9-benzyl-6-(4-chlorophenyl)-1,9-dihydropyrano[3,4-b]indole-3,4-dione;
(j) [1-benzyl-5-(4-chlorophenyl)-2-(hydroxymethyl)-1H-indole-3-yl](oxo)acetic acid;
(k) [1-benzyl-5-(1,1-biphenyl-4-yl)-2-(hydroxymethyl)-1H-indole-3-yl](oxo)acetic acid;
(l) 9-benzyl-6-(3-Methylphenyl)-1,9-dihydropyrano[3,4-b]indole-3,4-dione; or
(m) 9-benzyl-6-(1-1-bi-phenyl-4-yl)-1,9-dihydropyrano[3,4-b]indole-3,4-dione; or a pharmaceutically acceptable salt, solvate, or ester form thereof.

15. A method of treating muscle damage, muscle wasting, muscle degeneration, muscle atrophy or reduced rate of muscle repair, wherein said method comprises administering an effective amount of a compound of formula (XX), or a pharmaceutically acceptable salt, solvate or ester thereof, to a mammal in need thereof: 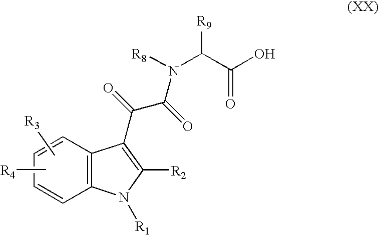
wherein:
R₁ is C₁-C₈ alkyl, C₃-C₆ cycloalkyl, —CH₂—C₃-C₆ cycloalkyl, pyridinyl, —CH₂-pyridinyl, phenyl or benzyl, the rings of the cycloalkyl, pyridinyl, phenyl and benzyl groups are optionally substituted by from 1 to 3 groups selected from the group consisting of halogen, C₁-C₆ alkyl, C₁-C₃ perfluoroalkyl, —O—C₁-C₃ perfluoroalkyl, C₁-C₃ alkoxy, —OH, —NH₂, and —NO₂;
R₂ is hydrogen, C₁-C₆ alkyl, C₃-C₆ cycloalkyl, —CH₂—C₃-C₆ cycloalkyl, or C₁-C₃ perfluoroalkyl;
R₃ is hydrogen, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂;
R₄ is phenyl, benzyl, benzyloxy, pyridinyl, or —CH₂-pyridinyl, wherein the rings of these groups are optionally substituted by 1 to 3 groups selected from the group consisting of halogen, C₁-C₃ alkyl, C₁-C₃ perfluoroalkyl, —O—C₁-C₃ perfluoroalkyl, C₁-C₃ alkoxy, —OH, —NH₂, and —NO₂;
R₈ is hydrogen, C₁-C₆ alkyl, C₃-C₆ cycloalkyl, —CH₂—C₃-C₆ cycloalkyl, C₁-C₃ perfluoroalkyl, aryl, substituted aryl, alkyl-aryl, or substituted alkyl-aryl; and
R₉ is hydrogen, C₁-C₆ alkyl, C₃-C₆ branched alkyl, C₁-C₆ hydroxyalkyl, 4-hydroxybenzyl, 3-indolylymethylene, 4-imidazolylmethylene, HSCH₂—, CH₃SCH₂CH₂—, H₂NC(═O)CH₂—, H₂NC(═O)CH₂CH₂—, HO₂CCH₂—, HO₂CCH₂CH₂—, H₂NCH₂CH₂CH₂CH₂—, H₂NC(═NH)NHCH₂CH₂CH₂—, or taken together with R₈, —CH₂CH₂CH₂—.

16. The method of claim 15 wherein the compound of formula (XX) is
(a) {[[1-(4-tert-butylbenzyl)-5-(3-methylphenyl)-1H-indol-3-yl](oxo)acetyl]amino}acetic acid;
(b) 2-[(2-{1-Benzyl-5-[4-(trifluoromethoxy)phenyl]-1H-indol-3-yl}-2-oxoacetyl)amino]acetic acid; or
(c) 2-[(2-{1-Benzyl-5-[3-(trifluoromethoxy)phenyl]-1H-indol-3-yl}-2-oxoacetyl)(methyl)amino]acetic acid; or a pharmaceutically acceptable salt or ester form thereof.

17. The method of claim 1, wherein said muscle damage, muscle wasting, muscle degeneration, muscle atrophy or reduced rate of muscle repair is caused by or associated with diabetes, hyperglycemia, motor neuron diseases, carpal tunnel syndrome, chronic infection, tuberculosis, Addison's disease, adult spinal muscular atrophy, anorexia nervosa, dermatomyositis, inclusion body myositis, incontinentia pigmenti, intercoastal neuralgia, juvenile rheumatoid arthritis, legg-calve-perthes disease, multifocal motor neuropathy, nephritic syndrome, osteogenesis imperfecta, post-polio syndrome, spinal muscular atrophy, nerve injury, neuropathy, diabetic neuropathy, or alcoholic neuropathy.

18. The method of claim 1, wherein said muscle damage is associated with normal muscle exertion or exercise.

19. The method of claim 1, wherein said muscle damage is associated with traumatic injury to muscle.

20. The method of claim 1, wherein said muscle wasting, muscle degeneration, muscular atrophy, or reduced rate of muscle repair is caused by or associated with muscular dystrophy.

21. The method of claim 20, wherein said muscular dystrophy is Duchenne's, Becker's, distal, ocular, Emery-Dreifuss, facioscapulohumeral, Fukuyama congenital, limb-girdle, myotonic, oculopharyngeal or severe childhood autosomal recessive.

22. The method of claim 21 wherein said muscular dystrophy is Duchenne's.

23. The method of claim 20, wherein said method further comprises the administration of at least one anabolic agent.

24. The method of claim 23, wherein said anabolic agent is an anabolic androgen.

25. The method of claim 20, wherein said method further comprises the administration of a glucocorticoid.
